(19) United States
(12) Patent Application Publication
(10) Pub. No.: US 2022/0213365 A1
(43) Pub. Date: Jul. 7, 2022

(54) SOY MILLING AND FRACTIONATION

(71) Applicant: CARGILL, INCORPORATED, Wayzata, MN (US)

(72) Inventors: Kevin R. ANDERSON, Cedar Rapids, IA (US); Flave Eugene MARKLAND, Jr., Atkins, IA (US)

(73) Assignee: CARGILL, INCORPORATED, Wayzata, MN (US)

(21) Appl. No.: 17/595,052

(22) PCT Filed: May 8, 2020
(86) PCT No.: PCT/US2020/032001

§ 371 Date: Nov. 8, 2021

Related U.S. Application Data
(60) Provisional application No. 62/846,284, filed on May 10, 2019.

(51) Int. Cl. C09J 189/00; C09J 11/06

(52) U.S. Cl. CPC C09J 189/00; C09J 11/06

(57) ABSTRACT

Various aspects disclosed relate to a soy product comprising from about 50.0 wt. % to about 60.0 wt. % dry protein, less than 35.0 wt. % carbohydrates, and an increased amount of protein in a dispersible fraction of the soy product, compared to the amount of protein in a dispersible fraction of a defatted soy flake having the same starting protein dispersibility index. The soy product can be made by a method of coarsely milling soy white flakes to provide a milled soy powder comprising one or more particles, each having a particle size of about 50 microns to about 100 microns at the 90th percentile mean and fractionating the milled soy powder to a soy product having greater than 50.0 wt. % dry protein, wherein the soy product comprises one or more particles, each having a particle size of about 20 microns to about 40 microns at the 90th percentile.

### BACKGROUND

Adhesive compositions have been made using raw natural materials such as starch, blood, and collagen extracts from animal bones and hides, milk protein and fish extracts, or soy beans. However, adhesive compositions made from these materials typically suffer from a number of drawbacks, including lack of durability and poor water resistance. Conventional soybean adhesives exhibit relatively high viscosity at a given solids level.

Phenol-formaldehyde and urea-formaldehyde adhesive resins were commonly utilized in adhesive compositions for use with composite wood products. Composite wood products made using phenol-formaldehyde and modified urea-formaldehyde resins have acceptable water resistance and are dominant in the exterior composite wood market. But these types of resins can cause large amounts of free formaldehyde to be released from the finished composite wood products.

In June of 2011, the U.S. Department of Health and Human Services listed formaldehyde as a “Known Human Carcinogen” on their “Report of Carcinogens” (ROC). As a result of these carcinogenic “classifications” for formaldehyde, new formulations of adhesives for wood products are desired.

### SUMMARY OF THE DISCLOSURE

The present disclosure provides, among other things, a soy product comprising: from about 50.0 wt. % to about 60.0 wt. % dry protein; less than 35.0 wt. % carbohydrates; and an increased amount of protein in a dispersible fraction of the soy product, compared to the amount of protein in a dispersible fraction of a defatted soy flake having the same starting protein dispersibility index (PDI). This soy product has several advantaged when used to formulate adhesives, including a high protein content and low carbohydrate content, including lower dispersible carbohydrate contents. Notably, when this soy product is formulated into an adhesive, it does not have the drawbacks associated with commercially available bio-based adhesives, instead having a relatively lower viscosity for a given solids content.

The present disclosure provides a soy product including from about 50.0 wt. % to about 60.0 wt. % dry protein; and less than 35.0 wt. % carbohydrates, wherein the soy product comprising one or more particles each having a particle size between about 20 microns and about 40 microns at the 90th percentile.

The present disclosure provides a soy product produced by a process, the process including the process including coarsely milling soy white flakes to provide a milled soy powder having a median 90th percentile particle size of about 50 microns to about 100 microns; and fractionating the milled soy powder to a soy product having greater than 50.0 wt. % dry protein, wherein the soy product having a mean 90th percentile particle size of about 20 microns to about 40 microns in various aspects.

The present disclosure provides an aqueous adhesive composition including the soy product and a cross-linker crosslinking the soy product in various aspects.

The present disclosure provides an engineered wood having at least one ply adhered together by the adhesive in various aspects.

The present disclosure provides a wood product comprising plywood, hardwood plywood, external grade plywood flooring, engineered wood flooring, high density fiber board, medium density fiber board, or particle board, wherein the wood product is adhered with the adhesive in various aspects.

The present disclosure provides an article including a particulate wood product comprising a plurality of wood particles, wherein the plurality of wood particles are adhered to each other by the adhesive in various aspects.

The present disclosure provides a method of making a soy product, the method including milling soy flakes to produce a milled soy product; and fractionating the milled soy powder to produce the soy product in various aspects.

The present disclosure provides for a high protein content soy based adhesive that is environmentally friendly and provides good tack (green adhesive strength), wet soak performance, and dry adhesive strength in various aspects.

### BRIEF DESCRIPTION OF THE FIGURES

The drawings illustrate generally, by way of example, but not by way of limitation, various aspects discussed in the present document.

FIG. 1 is a plot showing viscosity levels for pre-ground soy flour precursor materials 20 in Example 1.

FIG. 2 is a plot showing viscosity levels for unground precursor materials in Example 1.

FIG. 3 is a plot showing samples made from soy flake precursor material having a PDI of 50 in Example 1.

FIG. 4 is a plot showing particle size partitioning versus viscosity for samples in Example 1.

FIG. 5 is a plot showing RVA viscosity comparisons for pre-ground and ground samples in Example 1.

FIG. 6 is a plot showing percent changes in protein for samples in Example 1.

FIGS. 7-11 are plots showing particle size distribution of samples in Example 3.

FIG. 12 is a plot showing final viscosity of samples in Example 3.

FIG. 13 is a plot showing weight percent of samples in Example 4.

FIGS. 14-15 are plots showing viscosity over time for samples in Example 4.

FIGS. 16-23 are plots showing particle size distribution of samples in Example 4.

FIG. 24 is a plot showing 3-soak delamination results for samples in Example 5.

FIG. 25 is a plot showing the relationship between dispersible % solids and PDI value of the precursor material.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the subject matter is not intended to limit the claims to the disclosed subject matter.

A milled soy product with high protein content can be produced, for example, by a process including milling and particle size fractionation. The resulting milled soy product can be used as a base for a soy-type adhesive, with potential applications for wood products. Dried soy flakes can, for example, be ground and particle fractionated to produce a milled soy product with, for example, more than 50 wt. % dry protein. The soy product can, for example, be used to create an adhesive with low viscosity and high solids content, and other good adhesive properties.

Specifically, the dried soy flakes can be coarsely ground, by a grinder or milling process, and then fractionated out, such as by air classification or sieving. The coarse grinding and subsequent fractionation can allow for carbohydrate material and other impurities to be removed and increase the weight percentage of protein in the product, which is an important component in adhesive strength.

The produced soy product can be suspended in a solvent with a crosslinker, along with other appropriate adhesive components, to create an adhesive with low viscosity but high solids content. The adhesive can, for example, be used to create wood panels with minimized delamination and good bond strength.

### Soy Product

A soy product can be made from soy flake precursor material typically in a range of PDI (protein dispersibility index) from about 20 to about 90, such as a PDI 50, 70, or 90. PDI is a measurement of the degree the ground and/or defatted soy precursor material may be dispersed in water without particle settling. PDI can be determined, for example, by measuring the percentage of nitrogen in a sample that may be dispersed in water under standardized conditions. Several grades of commonly available soy flakes typically include PDI 90, 70, 50, and 20. Generally, the lower the PDI, the more the soy flake has been “toasted.” This results in higher PDI flakes having a white color.

The soy flakes can, for example, have a particle size of about 200 to about 1500 microns (e.g., about 500 to about 1000 microns). The soy flake precursor material is the material used for creation of the soy product. The soy flake precursor material can include, for example, 90 PDI, 70 PDI, 50 PDI, or 20 PDI flakes that can be ground and fractionated.

The soy flakes can be defatted. The fat may be removed from the soy flakes in a number of different methods. For example, the soy flakes can be defatted by using an organic solvent, such as hexane. Typically, the soy flakes can originate from dehulled seeds that are flattened into flakes, followed by the defatting with an organic solvent. The product of this defatting process can be commonly referred to as “white flakes.” In general, defatted soy flakes have about 1% or less fat by mass.

Specifically, in an example defatting process, the solvent can be extracted by passing the white flake through a chamber containing hot solvent vapor. Residual hexane can then, for example, be removed from soybean white flakes by passage through a chamber containing hexane vapor at a temperature less than about 75° C. Under such conditions, the bulk of the residual hexane vapor can volatize from the flakes and can be removed, for example, by a process such as vacuum extraction. This process can be referred to as “flash desolventized oilseed white flake.”

Alternatively, the soy flake product can be, for example, desolventized through a method referred to as “toasting.” In a toasting process, the hexane extracted flakes can be passed through a chamber containing steam at a temperature of at least about 105° C. The can, for example, cause the solvent in the flakes to volatize and be carried away with the steam. After desolventization, the flakes can be defatted. The chemical and physical properties of the soy precursor material can vary based on the previous processing and thermal history of the soy flakes or flour.

The precursor material can be ground to provide a coarsely ground soy material with a particle size larger than a conventional soy flour. For example, the precursor material can be coarsely milled through grinding or other milling mechanisms. The precursor material can be, for example, ground in a hand mixer to so that the soy product particles are on average larger than a conventional flour. Alternatively, the soy product precursor material can be milled in a machine, such as, for example, hammermill, a Retsch mill, a pin mill, a jet milling machine, or other milling machines as known to one in the art. The milling step can be typically done at an rpm from about 500 rpm to about 5500 rpm (e.g., about 800 rpm to about 1500 rpm). The speed at which the milling step occurs can vary depending on the size of the container, the volume of material, and the desired particle size, in addition to the impingement rate of the particle in the milling machine.

The milling step can typically produce milled soy products having a median particle size at the 90th percentile of about 50 microns to about 100 microns (e.g., less than 80 microns, or less than 60 microns, less than 40 microns, less than 30 microns or less than 25 microns; about 0.05 to about 90 microns, about 1 to about 50 microns, about 5 to about 30 microns, about 0.1 to about 10 microns or about 0.05 to about 5 microns). In some aspects, the milled soy product can contain a plurality pf particles, each particle having a largest dimension of less than 100 microns (e.g., less than 80 microns, or less than 60 microns, less than 40 microns, less than 30 microns or less than 25 microns; about 0.05 to about 90 microns, about 1 to about 50 microns, about 5 to about 30 microns, about 0.1 to about 10 microns or about 0.05 to about 5 microns). The particle size can be larger, for example, than a traditional soy flour, so that the milled soy product can be fractionated, and carbohydrates, hemi-cellulosic materials, or other impurities can be removed.

After coarsely milling the soy white flake precursor material, the milled material can be fractionated. Fractionation can be done, for example, by hand with a stack of sieves in series. For example, the stack can include, but is not limited to, sieves with 60 mesh size (pore per inch), 100 mesh size, 140 mesh size, 200 mesh size, and 325 mesh size. The milled material can be sieved through each sieve mesh size in series, ending with the 325 mesh size. The fractions of the material can be further sieved or used elsewhere. The material that successfully filters through the 325 mesh size can, for example, be used in adhesives.

Alternatively, the milled soy product can be fractionated with a machine such as an air classification system or similar. In this case, the milled soy product can be fractionated at speeds ranging from about 1000 rpm to about 2000 rpm (e.g., 1500 rpm). Alternative methods of fractionation, such as larger containers rotated at a lower rpm, could be used to separate the material.

The resulting milled and fractionated soy product can be used in adhesives. The resulting soy product can have, for example, a median particle size at the 90th percentile of about 20 microns to about 40 microns (e.g., about 25 microns to about 35 microns). In some aspects, the soy product can contain a plurality of particles, each particle having a largest dimension of less than about 40 microns (e.g., less than about 35 microns, or less than about 30 microns, or less than about 25 microns).

During the fractionation, undesirable materials are removed from the soy product, resulting a product having a dry protein percent of about 50.0 wt. % to about 60.0 wt. %, less than 35.0 wt. % carbohydrates, and more than 1.0 wt. % additional protein compared to the precursor material.

The soy product produced by milling and fractionation can, for example, include greater than 50.0 wt. % dry protein, preferably 51.0 wt. % protein (e.g., greater than 52.0 wt. % dry protein). The soy product can contain less than 60.0 wt. % dry protein.

Compared to the soy flake precursor material, the milled and fractionated soy product can have greater than 1.0 wt. % protein (e.g., greater than 2.0 wt. % protein, preferably greater than 3.0 wt. %).

The amount of dispersible particles in the soy product relative to the precursor material can be determined with the following formula (1):

Y=0.0568X+3.1261 (1)

In formula (1), Y is the amount of dispersible particles, and X is the PDI of the precursor material. The equation, calculated based on laboratory test data, has an R² value of 0.9974. The amount of dispersible protein can be determined as a percentage of the dispersible particles with methods known in the art.

The soy product can have more than 50.0 wt. % protein (e.g., more than 51.0 wt. % protein, or more than 52.0 wt. % protein, but in some aspects less than 60.0 wt. % protein). The soy product can contain, for example, less than 35.0 wt. % carbohydrates or preferably less than 34.0 wt. % carbohydrates (e.g., less than 33.0 wt. % carbohydrates). Protein is calculated with a nitrogen to protein factor of 5.71.

When suspended in solution, such as water, at a given solids content (% dry weight) of about 30 wt. % to about 50 wt. %, the soy product can have a viscosity lower than about 10,000 cPs (e.g., lower than about 50,000 cPs, lower than about 20,000 cPs, or lower than about 10,000 cPs).

### Adhesive

The dry milled soy product can be used to produce a wood adhesive composition for wood products such as an engineered wood containing at least one ply. In a typical process to produce the adhesive, a dry milled soy product, such as the soy product produced through milling and fractionating processes as described above, can be dispersed in water at a temperature of from about 15° C. to about 25° C. with sufficient mixing to produce a homogeneous protein dispersion at a target weight percent of solids to achieve the desired viscosity of the final wood adhesive composition to be applied to the wood (as further discussed below).

The wood adhesive composition can, for example, comprise from about 20 wt. % to about 60 wt. % dry solids, preferably from about 30 wt. % to about 50 wt. % dry solids, and more preferably from about 30 wt. % to about 40 wt. % by weight dry solids. These compositions can, for example, be used for the manufacture of engineered wood having at least one ply (for example from about 32 wt. % to about 37 wt. % dry solids can be common in wood adhesive composition for engineered wood products having at least one ply), and from about 40 wt. % to about 55 wt. % dry solids can be common in the wood adhesive composition for MDF (medium density fiberboard), HDF (high density fiberboard), particle board, and OSB (oriented strand board). The proportion of the wood adhesive composition not considered dry solids is water or other volatile solvents.

In conjunction with the solids content, the soy products described herein can have lower viscosity (and maintain a solids count) compared to conventional soy flour adhesives. Nonetheless, optionally, a viscosity reduction agent can be added to the composition, such as sodium bisulfite or sodium sulfite. For example, from about 0.1 wt. % to about 1.0 wt. % dry sodium sulfite based on the dry weight (solids content) of the soy product can be added to the adhesive composition.

Optionally, a defoamer can, for example, be added to control foam generation during the adhesive mixing process. After approximately 5 to 10 minutes of mixing the bisulfite or sulfite into the adhesive, the mixture typically can sufficiently react with the protein to reduce the dispersion viscosity.

Optionally, a plasticizing agent such as glycerol, can be added along with a protein unfolding agent such as urea. Typical addition levels of the plasticizing agent and the protein unfolding agent may be approximately 10 wt. % of the soy flour dry weight (solids content) plus the plasticizing agent and unfolding agent, if present, for each component.

After mixing to produce a thoroughly dispersed formulation, a crosslinker can be added. Many types of crosslinkers may be utilized. For example, the soy protein product may be crosslinked with reactive phosphorous oxide reagents such as phosphorous oxychloride, organophosphites, sodium trimeta-phosphate and similar. The soy protein product may also be crosslinked with poly(glycidyl ethers) such as neopentyl glycol diglycidyl ether for example, and their 1,2-hydroxyhalogen analogs. Other crosslinking reagents may be, for example, reactive polymers such as polyamidoamino epoxide (PAE) polymers. The soy protein product crosslinker may alternatively be an inorganic crosslinker. Generally, multivalent metal ion oxides, hydroxides, organo alkoxides, or halogen containing ions may be used. An example of an inorganic crosslinking agent is magnesium oxide.

For example, in the manufacture of multi-ply engineered wood, a PAE or a poly(glycidyl ether), (such as neopentylglycol diglycidyl ether, 1,4-butanediglycidyl ether, trimethylolpropane triglycidyl ether and other di- and tri-glycidyl ethers) can be added to obtain a wood adhesive composition having from 4 parts by dry weight (solids content) to 20 parts by dry weight (solids content) of the crosslinking agent to 100 parts by dry weight (solids content) of the protein flour containing 50.0 wt. % protein. Mixing can be continued until a uniform distribution of the crosslinking agent into the protein flour is achieved.

At this point, the pH of the wood adhesive composition can be adjusted to about 9 to 11, with a basic activating agent such as 25% sodium hydroxide solution. Optionally, the basic activating agent may be added to the dry protein flour before formulating to a dispersion with water in order to simplify the pH adjustment step. For example, dry magnesium oxide or calcium oxide can be added to a dry protein flour and mixed together thoroughly in a sufficient ratio such that when the protein oxide mixture is dispersed in water the pH of the formulation is raised to above pH 8. A typical mixture ratio can, for example, be 10 parts by weight of the protein product (i.e., containing 50.0 wt. % protein), to 1 part of the oxide.

Alternatively, the crosslinker can be added before or during the dispersion of the protein in the water. Alternatively, the basic activating agent can be added to water prior to the protein flour being added to the water. Other components used in the wood adhesive composition can, for example, be added simultaneously with the protein flour or after the protein flour has been dispersed in the water.

When the desired components of the wood adhesive composition are sufficiently mixed, and the desired pH of the formulation has been achieved, the wood adhesive can be ready to be applied to a wood product.

In the manufacture of engineered wood products having at least one ply, for example, a preferable wood adhesive composition using the milled and fractionated soy product described above could, for example, have a viscosity ranging from about 10,000 cPs to about 100,000 cPs (e.g., about 20,000 cPs to about 50,000 cPs). The adhesive formulated from the milled and fractionated soy product can, for example, have a corresponding solids content of about 30 wt. % to about 50 wt. % (e.g., about 35 wt. % to about 45 wt. %). This viscosity range is lower than conventional wood adhesives but maintained a high solids content, increasing adhesive properties of the adhesive formulation.

Engineered wood panels can be made using the wood adhesive compositions by applying the adhesive to the wood layers using any of several commercial processes; including roll coaters, curtain coaters and spray coaters. The engineered wood product can typically have a core and one or more plies. The core material can be, for example, a wood or engineered wood core. The one or more plies can be a hardwood or softwood attached to the core. For example, the engineered wood product can contain core or ply components made of fir, maple, oak, or other appropriate types of veneers.

The assembled engineered wood panel having at least one ply can then be subjected to either a cold and/or hot pressing process to complete the adhesive transfer and curing, to form a durable wood bond. The press times to produce the final engineered wood product typically can range from only a few seconds to several minutes depending on the press temperature and thickness of the panel being pressed. The press temperatures typically can range from room temperature to about 165° C. The press pressures typically can range from about 25 psi to about 200 psi, more preferably from about 50 psi to about 175 psi (e.g., from about 75 psi to about 150 psi). The temperatures and press pressures used can vary based on the final pH of the wood adhesive composition, the protein and crosslinker utilized, the wood type utilized, the moisture content of the wood and the overall wood composite product thickness. Achieving a temperature in the middle of the product (that is typically in the form of a panel) of about 90° C. to about 105° C. for about 30 to about 60 seconds is generally sufficient to cure the adhesive to achieve the desired panel properties.

When the composite wood product comprises multi-ply engineered wood, the veneers utilized to manufacture the engineered wood typically can have moisture levels from about 2 wt. % to about 12 wt. % moisture. In some aspects it is preferable that the veneers utilized to manufacture the engineered wood have from 5 to 10 wt. % moisture, preferably from about 7 wt. % to 9 wt. % moisture, and more preferably from about 6 wt. % to about 9 wt. % moisture. These moisture levels can potentially enhance the ability of the finished cured multi-ply engineered wood to pass soak test requirements.

Other composite wood products, such as particleboard (PB) or medium density fiberboard (MDF), high density fiberboard (HDF) and oriented strand board (OSB) can also be produced using the wood adhesive compositions using the soy product described in detail above. In any of those products, the adhesive typically can be applied to the wood fiber or particles using any of the commercially viable adhesive application processes including spraying, paddle shear mixing, and blow-line, and other processes known of skill in the art to form an adhesive impregnated wood mat. The uncured wood mat can then be compressed using a cold press and then placed in a hot press similarly as described, above for the manufacture of engineered wood. A continuous press may also be used. Variations of the wood adhesive composition expected to be utilized to produce composite wood product panels of this type typically can be of lower viscosity (e.g., less than about 30,000 cPs at 80° F. (27° C.)) (for example less than about 20,000 cPs at 80° F. (27° C.), and preferably less than about 10,000 cPs at 80° F. (27° C.)) and higher solids (about 45% total solids).

The adhesive can optionally be used to produce other products including, but not limited to, plywood, hardwood plywood, internal grade wood flooring, engineered wood flooring, HDF, MDF, or particle board as described above. The adhesive can have, for example, bond lines that are substantially maintained after a standard 3-cycle soak test and have substantially no delamination after 3-soak cycle testing.

Overall, a milled and fractionated soy product can be used to create adhesives, for example, to be used in wood products. The soy product can have a higher protein level over standard commercial soy flours and can produce and adhesive with a lower viscosity but higher solids content. The adhesive can perform well with wood products, exhibiting low delamination and strong bond lines.

### Definitions

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “cure” as used herein refers to exposing to radiation in any form, heating, or allowing to undergo a physical or chemical reaction that results in hardening or an increase in viscosity.

The term “solvent” as used herein refers to a liquid that can dissolve a solid, liquid, or gas. Non-limiting examples of solvents are silicones, organic compounds, water, alcohols, ionic liquids, and supercritical fluids.

The term “room temperature” as used herein refers to a temperature of about 15° C. to 28° C.

The term “standard temperature and pressure” as used herein refers to 20° C. and 101 kPa.

The term “dry weight” or “solids content” refers to a measure of the components of the composition absent water. For example, if the protein comprises 50% dry weight of the wood adhesive composition, then the protein makes up 50% of the composition remaining after any water present in the composition has been excluded from the calculation.

### EXAMPLES

Various aspects of the present disclosure can be better understood by reference to the following Examples which are offered by way of illustration. The present disclosure is not limited to the Examples given herein.

### Example 1—Laboratory Grinding and Fractionating

Two types of precursor materials were used for comparison: unground soy flakes (which were coarsely ground in laboratory) and pre-ground soy flours (which were ground down to flours prior to usage in Example 1). Unground soy flake precursor materials included extracted and dried soy white flakes (WF) with protein dispersibility index (PDI) of 90 PDI soy white flake (“90 PDI WF”) and 20 PDI soy white flake (“20 PDI WF”), in addition to a soy Extracted Meal flake (“Meal WF”) taken from the meal feed line, before milling, and before removal of hexane and air dried. (Cargill®, Cedar Rapids, Iowa). Additionally, a 50 PDI soy white flake (“50 PDI WF”) that was not yet milled was used. (ADM facility, Amsterdam). These samples were not yet ground or milled when obtained.

Pre-ground soy flour precursor materials (i.e., soy flours) included standard products of 100 mesh and 200 mesh, with PDI values of 90, 70, and 20 (e.g., “100/90,” “100/70,” “100/20,” “200/90,” “200/70,” “200/20” samples) (Cargill®, Cedar Rapids, Iowa). Additionally, an experimental lab sample of 300 mesh 90 PDI soy flour (300/90) was used. All of the precursor materials are summarized in Table 1.

Example 1 included manufacture of a soy product through steps of first milling (grinding) and then fractionating (sieving) of the following samples shown in Table 1:

TABLE 1
Sample Descriptions for Example 1.

| Sample ID | Type | Precursor Material Description |
|---|---|---|
| 20 PDI WF | Unground | Soy Flakes |
| | | 20 PDI |
| 90 PDI WF | Unground | Soy Flakes |
| | | 90 PDI |
| Meal WF | Unground | Extracted Meal Flake |
| 50 PDI WF | Unground | Soy Flakes |
| | | 50 PDI |
| 100/90 | Ground | Commercially Available Soy Flour |
| | | 100 Mesh, 90 PDI |
| 100/70 | Ground | Commercially Available Soy Flour |
| | | 100 Mesh, 70 PDI |
| 100/20 | Ground | Commercially Available Soy Flour |
| | | 100 Mesh, 20 PDI |
| 200/90 | Ground | Commercially Available Soy Flour |
| | | 200 Mesh, 90 PDI |
| 200/70 | Ground | Commercially Available Soy Flour |
| | | 200 Mesh, 70 PDI |
| 200/20 | Ground | Commercially Available Soy Flour |
| | | 200 Mesh, 20 PDI |
| 300/90 | Ground | Commercially Available Soy Flour |
| | | 300 Mesh, 90 PDI |

Because the ground precursor materials (100/90, 100/70, 100/20, 200/90, 200/20, 300/90) were already flours when obtained, they were not further ground.

For the 20 PDI WF, 90 PDI WF, Meal WF, and 50 PDI WF samples, unground soy flake precursor materials were coarsely ground with a small coffee double blade grinder (Krups) with a capacity of about 50 grams. The soy flakes loaded into the coffee grinder were about 50 g, or level to the top of the grinder base (for low density materials). A plastic cover was added prior to grinding, which doubled the capacity of the grinding operation. Once grinding had begun, the soy white flakes circulated within the grinder and the cover. Grinding was done for approximately one minute for each sample to produce a milled soy product that was then transferred to screens for sieving (fractionation).

The ground soy product (all samples, either ground in laboratory or received as a pre-ground flour precursor material) was then sieved (i.e., fractionated) with a series of standard sieve screens of successively smaller size ranges. Sieve fractionation was performed by hand. About three loads of milled soy product were loaded onto a sieve for fractionating at a time. The sieve series stack included 60 mesh, 100 mesh, 140 mesh 200 mesh, and a 325 mesh rating screens (W.S. Tyler Inc.). The 60 mesh screen sifted a fraction of 60 mesh to 100 mesh size particles (e.g., about 250 to about 150 microns); the 100 mesh screen sifted a fraction of 100 to 140 mesh size particles (e.g., about 150 to about 104 microns); the 140 mesh screen sifted a fraction of 140 mesh to 200 mesh size particles (e.g., about 105 to about 74 microns); the 200 mesh product sifted a fraction of 200 mesh to 325 mesh size particles (e.g., about 74 to about 44 microns); and the 325 mesh product was for materials less than about 44 microns in size.

Sieving of the milled soy product was progressively more difficult for each smaller sieve size. Van der Waal forces held smaller fractionations of the milled soy product together, resulting in slower sieving of those fractions and blockage on the sieve screens, which was broken up through vigorous movement such as a shaking. Course material that did not filter through the initial 60 mesh screen was considered “overs.” When a pure fractionation of overs was reached on the screen, the material flowed across the screen smoothly. After this occurred, that individual mesh screen would be removed from the sieve stack, and the smaller mesh screens would continue to be used in series. This was repeated until all screens, down to the 325 mesh rating, were used, and a final fractionated soy product was produced. Overs for each mesh size could be separated and studied.

### Example 2—Analysis of Example 1

The resulting soy products that began as an unground soy flake precursor material (20 PDI WF, 90 PDI WF, Meal WF, and 50 PDI WF samples) produced about 40 wt. % to about 50 wt. % total soy product compared to the precursor material. In contrast, the soy product that began as pre-ground soy flour precursor materials (100/90, 100/70, 100/20, 200/90, 200/20, 300/90 samples) produced about 70 wt. % to about 84 wt. % compared to the precursor soy flour.

However, the soy product that began as pre-milled soy flour precursor materials did not produce as great of a protein amount compared to the soy product that began as unground soy flake precursor materials and was coarsely ground in the laboratory. It is theorized that the finely ground soy flour precursor material also contained finely ground fiber and hemi-cellulosic material that are considered impurities. In general, a higher protein level in soy flour allows for stronger adhesive properties.

The resulting soy product, after milling and fractionation, was analyzed for a variety of properties, including particle size, protein and carbohydrate content, and viscosity, which are summarized in Tables 2-7 below.

TABLE 2
100/90 Commercially Available Flour Sample Analysis.

| Sample | RVA | Mass Grams | % Fraction | % Pr Dry |
|---|---|---|---|---|
| 100/90 Flour | 401 | — | — | 49.29 |
| 100/90 60M | — | 1.48 | 3.10 | — |
| 100/90 100M | 3271 | 3.16 | 6.61 | — |
| 100/90 140M | 5040 | 3.58 | 7.49 | — |
| 100/90 200M | 4306 | 5.88 | 12.30 | — |
| 100/90 325M+ | 237 | 33.7 | 70.50 | 50.85 |
| | Total | 47.8 | 100.00 | — |

Table 2 shows viscosity analysis (RVA), mass (Mass Grams), mass fraction (% Fraction) and amount of protein dry (% Pr Dry) for the 100/90 commercially available flour (Flour+) at each sieve in the sieve stack during fractionation at mesh (M) sizes 60, 100, 140, 200, and 325+, in addition to the starting Flour. Each of the mesh (M) sizes represents the material that did not filter though that mesh (i.e., the 100/90 60M sample includes the flour that sat on top of the 60M sample). The 325M+ sample describes material that filtered all the way through the 325M size.

TABLE 3
200/90 Commercially Available Flour Sample Analysis

| Sample | RVA | Mass Grams | % Fraction | % Pr Dry |
|---|---|---|---|---|
| 200/90 Flour | 407 | — | — | 49.71 |
| 200/90 60M | — | 0.04 | 0.08 | 39.81 |
| 200/90 100M | — | 1.03 | 2.06 | 33.01 |
| 200/90 140M | 3342 | 2.24 | 4.48 | 36.51 |
| 200/90 200M | 6005 | 4.5 | 8.99 | 42.62 |
| 200/90 325M+ | 346 | 42.22 | 84.39 | 51.65 |
| | Total | 50.03 | 100.00 | — |

Table 3 shows viscosity analysis (RVA), mass (Mass Grams), mass fraction (% Fraction) and amount of protein dry (% Pr Dry) for the 200/90 commercially available flour (Flour+) at each sieve in the sieve stack during fractionation at mesh (M) sizes 60, 100, 140, 200, and 325, in addition to the starting Flour. Each of the mesh (M) sizes represents the material that did not filter though that mesh (i.e., the 100/90 60M sample includes the flour that sat on top of the 60M sample). The 325M+ sample describes material that filtered all the way through the 325M size.

TABLE 4
Meal WF Sample Analysis.

| Sample | RVA | Mass Grams | % Fraction | % Pr Dry |
|---|---|---|---|---|
| Meal WF | — | — | — | 48.92 |
| Meal WF Overs | — | 35 | 25.93 | 46.27 |
| Meal WF 60M | 3386 | 17.6 | 13.04 | 48.53 |
| Meal WF 100M | 3559 | 12.9 | 9.56 | 48.14 |
| Meal WF 140M | 3467 | 8.6 | 6/37 | 47.94 |
| Meal WF 200M | 3236 | 12.18 | 9.02 | 45.97 |
| Meal WF 325M+ | 177 | 48.72 | 36.09 | 52.07 |
| | Total | 135 | 100.00 | — |

Table 4 shows viscosity analysis (RVA), mass (Mass Grams), mass fraction (% Fraction), and amount of protein dry (% Pr Dry) for the meal white flake (Meal WF, a soy flake taken out of the “soy meal” line) sample after grinding and at each sieve in the sieve stack during fractionation at mesh (M) sizes 60, 100, 140, 200, and 325. “Overs” are the material that did not go through the sieve stack. Each of the mesh (M) sizes represents the material that did not filter though that mesh (i.e., the Meal WF 60M sample includes the flour that sat on top of the 60M sample). The 325M+ sample describes material that filtered all the way through the 325M size. The meal flake was a soy flake taken out of a processing line for creating a standard soy meal, prior to milling of that soy flake.

TABLE 5
50 PDI WF Sample (ground in lab) Analysis.

| Sample | RVA | Mass Grams | % Fraction | % Pr Dry |
|---|---|---|---|---|
| 50 PDI WF | — | — | — | 47.84 |
| 50 PDI WF Overs | — | 35.65 | 33.84 | — |
| 50 PDI WF 60M | 3826 | 12.54 | 11.90 | 47.36 |
| 50 PDI WF 100M | 26.17 | 7.8 | 7.40 | 48.50 |
| 50 PDI WF 140M | 1906 | 3.7 | 3.51 | 49.29 |
| 50 PDI WF 325M | 399 | 45.67 | 43.35 | 50.19 |
| | Total | 105.36 | 100.00 | — |

Table 5 shows viscosity analysis (RVA), mass (Mass Grams), mass fraction (% Fraction) amount of protein (% Pr As Is), and amount of protein dry (% Pr Dry) for the 50 PDI white flake (50 PDI WF) sample after grinding and at each sieve in the sieve stack during fractionation at mesh (M) sizes 60, 100, 140, and 325. “Overs” are the material that did not go through the sieve stack. Each of the mesh (M) sizes represents the material that did not filter though that mesh (i.e., the 60M sample includes the flour that sat on top of the 60M sample). The 325M+ sample describes material that filtered all the way through the 325M size.

TABLE 6
90 PDI WF Sample (ground in lab) Analysis.

| Sample | RVA | Mass Grams | % Fraction |
|---|---|---|---|
| 90 PDI WF Overs | — | 36.7 | 31.02 |
| 90 PDI WF 60M | 3126 | 14.43 | 12.20 |
| 90 PDI WF 100M | 3091 | 10 | 8.45 |
| 90 PDI WF 140M | 2520 | — | — |
| 90 PDI WF 200M | 3090 | 7.8 | 6.59 |
| 90 PDI WF 325M | 296 | 49.37 | 41.73 |
| | Total | 118.3 | 100.00 |

Table 6 shows viscosity analysis (RVA), mass (Mass Grams), and mass fraction (% Fraction) for the 90 PDI white flake (90 PDI WF) sample after grinding and at each sieve in the sieve stack during fractionation at mesh (M) sizes 60, 100, 140, 200, and 325. “Overs” are the material that did not go through the sieve stack. Each of the mesh (M) sizes represents the material that did not filter though that mesh (i.e., the 60M sample includes the flour that sat on top of the 60M sample). The 325M+ sample describes material that filtered all the way through the 325M size.

TABLE 7
90 PDI WF Sample (ground in lab) Sieving Analysis.

| Milling step | Fractionation | % Pr DB |
|---|---|---|
| 90 PDI White Flake Initial | — | 49.70 |
| On #12 Mesh | Screened | 36.49 |
| Thru #12 Mesh | Screened | 46.94 |
| On #18 Mesh | Screened | 46.94 |
| On #20 Mesh | Screened | 50.10 |
| On #35 Mesh | Screened | 50.95 |
| Thru #35 Mesh | Screened | 50.98 |
| On #100 Mesh | Screened | 50.07 |
| Thru #100 Mesh | Screened | 51.20 |
| On #200 Mesh | Screened | 48.98 |
| Thru #200 Mesh | Screened | 52.21 |
| On #325 Mesh | Screened | 47.66 |
| Thru #325 Mesh | Screened | 53.33 |

Table 7 shows the 90 PDI white flake (90 PDI WF) sample after grinding and at each sieve in the sieve stack during fractionation. Table 7 illustrates the protein dry (% Pr DB) for the 90 PDI WF sample.

The particle sizes of the resulting soy products was shown by image analysis. A large portion of the resulting soy product was less than about 20 microns in size. Mass balance of the resulting soy product was measured and calculated as a percent fraction of the total weight.

The resulting soy product was mixed into water to form a water/flour suspension. The viscosity of these suspensions was analyzed on a Rapid Visco Analyzer (RVA) (Perten, Sweden). To analyze viscosity, 6 g of the produced soy product was added to a Teflon® coated aluminum RVA cup. Two drops (e.g., about 0.02 g) of defoamer (MCA270, Hydrite, Inc.) was added, followed by 10 drops (e.g., about 0.2 g) of a 20% sodium sulfite solution. Then, 20 g deionized (DI) water was added, followed by 5 drops of 25% NaOH (e.g., about 0.3 g) to bring the suspension to a pH of about 10.2. The mixture was then brought to 30 g with additional DI water (e.g., about 20% total dry solids). A round spatula was used to mix the suspension until the soy flour was hydrated. The RVA plastic mixing top was added to the sample cup and the unit placed in the RVA viscometer. An RVA program of about 5 minutes was used at about 27° C., including a one minute mixing time at about 500 rpm, followed by a measurement speed of 100 rpm for 4 minutes. At the end of the cycle, the viscosity reading was recorded. Measurements were performed in duplicate and averaged. Measurement precision between replicates was about less than 10%.

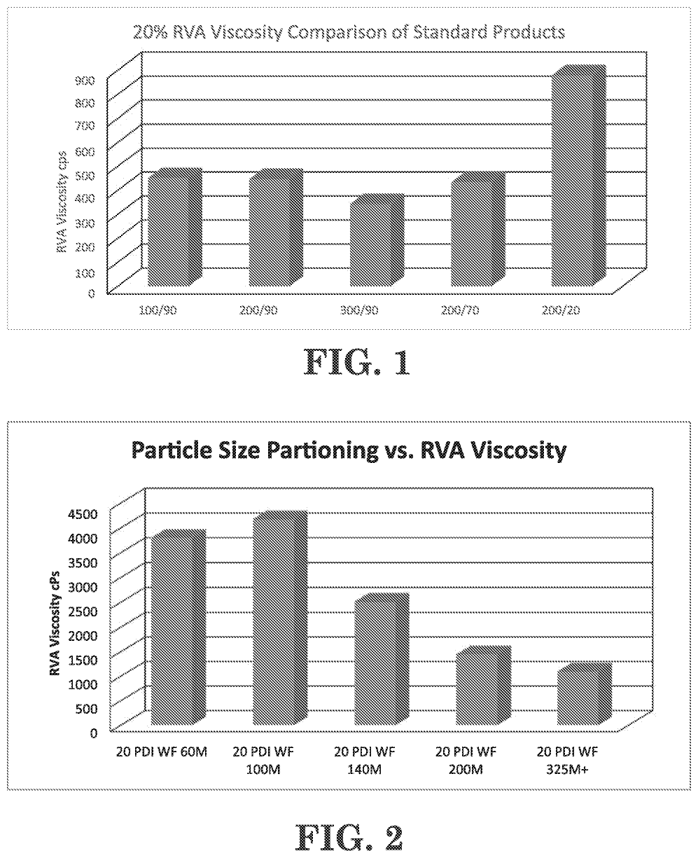

Prior to fractionation, commercially available pre-ground soy flour precursor materials (100/90, 100/70, 100/20, 200/90, 200/20, 300/90) did not show decreased viscosity. Both commercial fine grinding of soy flour precursor materials and PDI (protein dispersibility index) levels had minimal effect on the suspension viscosity. Under PDI levels of 20, the viscosity of the samples was increased to several times the viscosity of 90 PDI or 70 PDI samples. FIG. 1 shows the comparison of RVA viscosity levels (“RVA Viscosity cps”) for pre-ground soy flour precursor materials having mesh/PDI ratings of 100/90, 200/90, 300/90, 200/70, and 200/20.

Produced soy products using un-ground soy flake precursor materials (20 PDI WF, 90 PDI WF, Meal WF, and 50 PDI WF samples) showed more variation in viscosity. The samples using a white flake with 20 PDI, which were ground and sieved in laboratory, did not respond as well to fractionation compared to white flakes with higher PDI values. For example, the viscosity of the white flake 20 PDI sample was significantly higher compared to other white flake products processes in the same manner. FIG. 2 shows RVA viscosity levels (“RVA Viscosity cps”) for white flake (unground precursor material) having PDI/mesh values of 20/60 (“20 PDI WF 60M”), 20/100 (“20 PDI WF 100M”), 20/140 (“20 PDI WF 140M”), 20/200 (“20 PDI WF 200M”), and 20/325 (“20 PDI WF 325M+”).

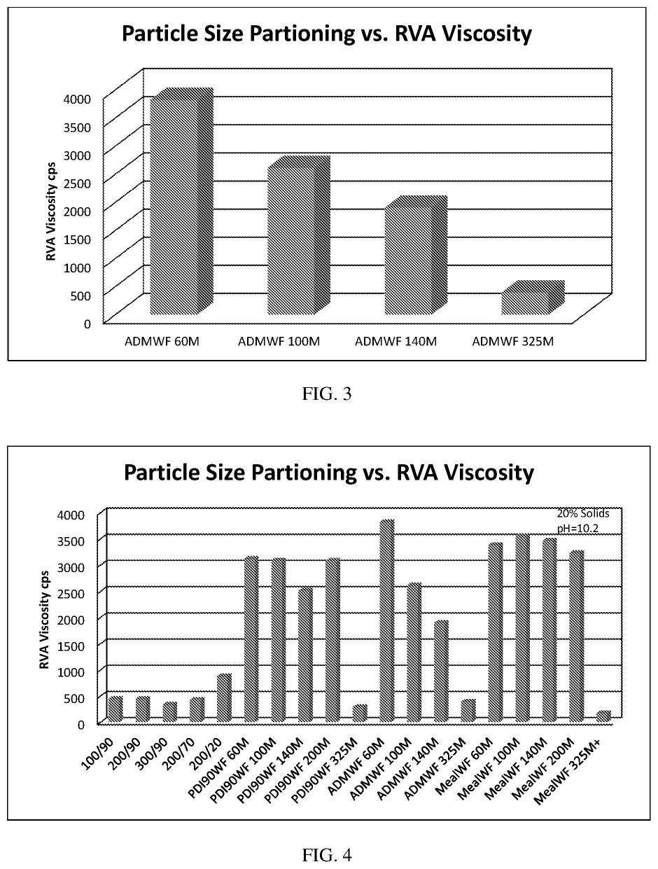

However, products using un-ground soy flake precursor materials having higher PDI values allowed for lower viscosity when fractionated down to the 325 mesh size. For example, FIG. 3 shows samples made from soy white flake precursor material having a PDI of 50 at 60 mesh (“ADMWF 60M”), 100 mesh (“ADMWF 100M”), 140 mesh (“ADMWF 140M”), and 325 mesh (“ADMWF 325M”). The 325 mesh size particles from this sample had a much lower viscosity (“RVA Viscosity cps”) compared to both other fractionations of this particular sample and compared to the 20 PDI samples in FIG. 2. This indicates a break point between 20 PDI and 50 PDI where the usefulness of screening to obtain the 325 fraction is minimal.

Overall, the 325 mesh product for all samples was substantially lower in RVA suspension viscosity compared to larger fractions of the product. These results are summarized in FIG. 4 (showing viscosity levels “RVA Viscosity cps” at 20% solids), comparing both using pre-ground precursor materials and samples produced using unground precursor materials. Shown are pre-ground precursor materials with mesh/PDI values of 100/90, 200/90, 300/90, 200/70, 200/20; laboratory ground samples (i.e., made from unground precursors) first set of white flakes (90 PDI WF) with mesh/PDI values of 60/90, 100/90, 140/90, 200/90, 325/90, second set of white flakes (50 PDI WF) with mesh values of 60, 100, 140, 325, and third set of white flakes (Meal WF) with mesh values of 60, 100, 140, 200, and 325.

When pure size fractions were obtained, such as the 90 PDI WF and Meal WF samples, the higher viscosity of the smaller sieve sizes (i.e., the larger particles) was relatively constant until the 325 mesh product was obtained.

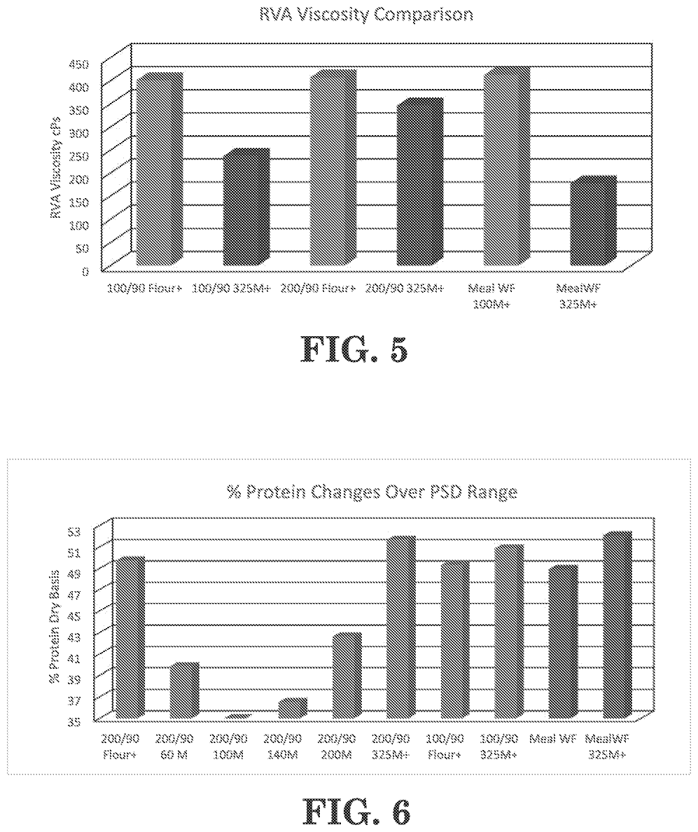

Samples produced using pre-ground soy flour precursor materials were compared to the samples produced using unground soy flake precursor materials. For samples using pre-ground soy flour precursor materials, (100/90, 100/70, 100/20, 200/90, 200/20, 300/90), the samples using 325 mesh fraction sizes had lower viscosity compared to samples using 100 mesh or 200 mesh sizes. The differences in viscosity between these fractionation sizes was minimal after fine grinding, presumably due to the grinding of impurities such as hemi-cellulosic material. A summary of these test is shown in FIG. 5 (showing viscosity levels “RVA Viscosity cPs” for flour (Flour+) samples, 325 mesh (325+) samples, and meal white flake (Meal W F) samples). These results indicate that the small particle fraction dominates the viscosity of these fractions.

The percent of protein was measured using a LECO® Nitrogen Analyzer (LECO, St. Joseph, Mich.). Duplicate and triplicate determinations were performed on each individual sample. Precisions between replicates was less than about 5%. The percent of protein measured was corrected for moisture as determined in the Mettler® Moisture Balance (MSSC Direct, Toledo, Ohio). A Nitrogen to Protein factor of 5.71 was used to calculate the % Protein.

The percent of protein (% Protein Dry Basis) gains in the fractionation experiments was varied when comparing the samples from the unground precursor materials (20 PDI WF, 90 PDI WF, Meal WF, and 50 PDI WF samples) to the samples from the pre-ground precursor materials (100/90, 100/70, 100/20, 200/90, 200/20, 300/90 samples). This is shown in FIG. 6. As the difference size fraction are purified down the sieve stack, the percent of protein in the sample varies considerably, resulting in a higher protein level for the 325 mesh products. For example, in the meal (Meal WF) samples, the percent of protein gain is significant, rising to 52.0% protein on a dry basis.

In general, the protein increase for samples processed from ground soy flours (100/90, 100/70, 100/20, 200/90, 200/20, 300/90 samples) are not as significant as the protein increase observed from samples starting from un-ground soy flake (20 PDI WF, 90 PDI WF, Meal WF, and 50 PDI WF samples). Overall, Example 1 shows the use of coarsely milled and fractionated soy product samples can be produced in laboratory having good protein content compared to conventional soy flours.

### Example 3—Jet Milling Trial

Milled and fractionated soy products were also produced with jet milling and air classification technology. Examples 3 and 4 used a PDI 90 white flake (“90 PDI WF”) precursor material (Cargill, Cedar Rapids, Iowa).

Example 3 included the use of varying speeds to grind the soy flake material, and analysis of protein content and viscosity. The results of Example 3 analyzed milling and fractionating of 90 PDI white soy flakes (“90 PDI WF”). The 90PDI WF was defatted (i.e., hexane was extracted) but had not been previously milled. In Example 3, the 90PDI WF was coarsely milled and fractionated to reduce the amount of non-proteinaceous components.

For Example 3, 90 PDI WF was used. (Cargill®, Cedar Rapids, Iowa). Equipment used for Example 3 included a Model DPM-2 Fluidized Bed Jet Mill for jet milling, and a Model 250 High Efficiency Centrifugal Air Classifier for air classification (Aveka CCE Technologies, Cottage Grove, Minn.).

Particle size was analyzed with a Retsch Technology CamsizerXT instrument. (Retsch Technology GmbH, Haan, Germany). For particle size, dispersed particles passed in front of two bright, pulsed LED light sources. The shadows of the particles were captured with two digital cameras. One camera was optimized to analyze the small particles with high resolution, the other camera detected the larger particles with good statistics, due to a large field of view. Each camera was illuminated by one LED with optimized brightness, pulse length and field of illumination. To cover a small measuring window of limited space with two light sources, optics and cameras, the optical paths of both cameras intersected in the measurement area. Particle size and particle shape were analyzed with software which calculated the respective distribution curves in real time.

Viscosity measurements (in centipoise) were done at a constant high sheer rate of 15 l/s with an Anton Paar MCR-101 rheometer using 50 mm parallel plate test geometry held at 25° C. with a 1 mm gap. Rotational viscosity was measured with a Brookfield DV-1 viscometer with a #6 or #7 spindle at 10 rpm.

With the Brookfield DV-1 viscometer, for the viscosity shear, the sample was rapidly hand sheared in the container for a period of 30 seconds to provide the resin sample with high shear with minimal air entrapment. Immediately after hand shearing, the spindle was lowered into the sheared solution. After a 10 second wait, the motor was turned on and readings began after 10 seconds and 60 seconds of run time. For viscosity recovery, the sample was prepared, and viscosity measured at a constant time of 10 seconds and measured at various recovery times.

Eight different jet milling and air classification conditions were evaluated. Three jet milling parameters were controlled during sample processing. First, the feed rate of the sample into the DPM-2 Jet Mill from a feed hopper was controlled. Second, fluid bed inlet pressure into the DPM-2 Jet Mill was controlled to keep the sample suspended within the unit. Third, the inlet air fan speed into the DPM-2 unit was controlled to monitor the amount of energy being injected into the system. These parameters were controlled in part due to higher rpms (i.e., more energy) resulting in smaller particle sizes.

Feed rate in the DPM-2 unit was monitored by internal static pressure to prevent too high of a product feed rate. The operator adjusted the feed rate, air pressure, and inlet fan speed to maintain internal static pressure in a steady state. Sampling occurred during these adjustments while particle size was monitored.

After jet milling, some samples were subjected to air classification. For air classification, three primary components were controlled. First, product feed into the air classifier was controlled by a vibratory hopper. Inlet air pressure and rotor speed were maintained to keep particles suspended but not allow product to be pushed in the wrong direction. If the classifier was fed too much product, coarse material would be mixed with the fine material. If fed too slowly, the opposite would occur. The Samples run in Example 3 are summarized in Table 8 below. “Overs” are the wasted material not use, while “fines” are the final fractionated material.

TABLE 8
Summary of Example 3.

| | | | | | Q3 | Q3 | Q3 | |
|---|---|---|---|---|---|---|---|---|
| | | | | | %10 | %50 | %90 | Mean |
| Sample | Precursor | Jet | Air | % | Part. | Part. | Part. | Particle |
| ID | Material | Milling | Classified | Protein | Size | Size | Size | Size μ |
| Control | Example 1 | — | — | — | 5.2 | 11.62 | 27.55 | 14.33 |
| Control | 90 PDI | — | — | — | 22.64 | 278.58 | 1122.75 | 405.86 |
| | WF | | | | | | | |
| 1 | 90 PDI | 500 rpm | 700 rpm | 51.25 | 6.83 | 29.53 | 67.84 | 34.62 |
| | WF Fines | | | | | | | |
| 2 | 90 PDI | 500 rpm | 900 rpm | 44.59 | 51.37 | 104.11 | 179.29 | 113.16 |
| | WF Overs | | | | | | | |
| 3 | 90 PDI | 500 rpm | 900 rpm | 51.99 | 5.83 | 15.95 | 43.70 | 21.29 |
| | WF Fines | | | | | | | |
| 4 | 90 PDI | 500 rpm | 1500 rpm | 46.86 | 22.09 | 56.47 | 146.30 | 72.21 |
| | WF Overs | | | | | | | |
| 5 | 90 PDI | 500 rpm | 1500 rpm | 52.68 | 5.11 | 10.54 | 25.94 | 13.55 |
| | WF Fines | | | | | | | |
| 6 | 90 PDI | 5200 rpm | — | 52.05 | 3.71 | 6.12 | 9.54 | 6.73 |
| | WF Fines | | | | | | | |
| 7 | 90 PDI | 1500 rpm | — | 51.53 | 5.40 | 13.24 | 35.42 | 17.31 |
| | WF Fines | | | | | | | |
| 8 | 90 PDI | 500 rpm | — | 50.36 | 6.06 | 19.70 | 66.03 | 31.05 |
| | WF | | | | | | | |
| | Coarse | | | | | | | |

Table 8 shows the speeds at which each sample was jet milled and air classified (in rpm) and compares the particle size for each sample (in microns) to both an unprocessed 90 PDI WF and the averages of laboratory made Example 1 with 90 PDI WF. The jet milled 500 rpm and air classified 1500 rpm sample and the jet milled only 1500 rpm samples had the best match for Q3 90% of particle size. The jet milled 5200 rpm sample had the higher protein content and the smallest particle size. During Example 3, the question was whether the air classification removed the non-protein material from the samples.

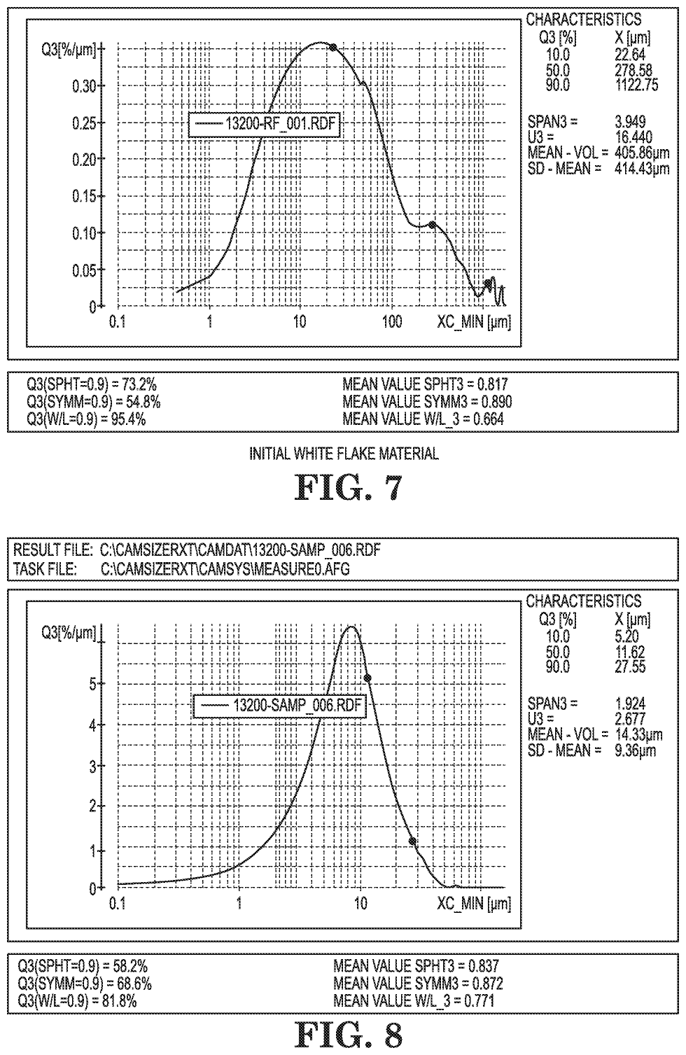

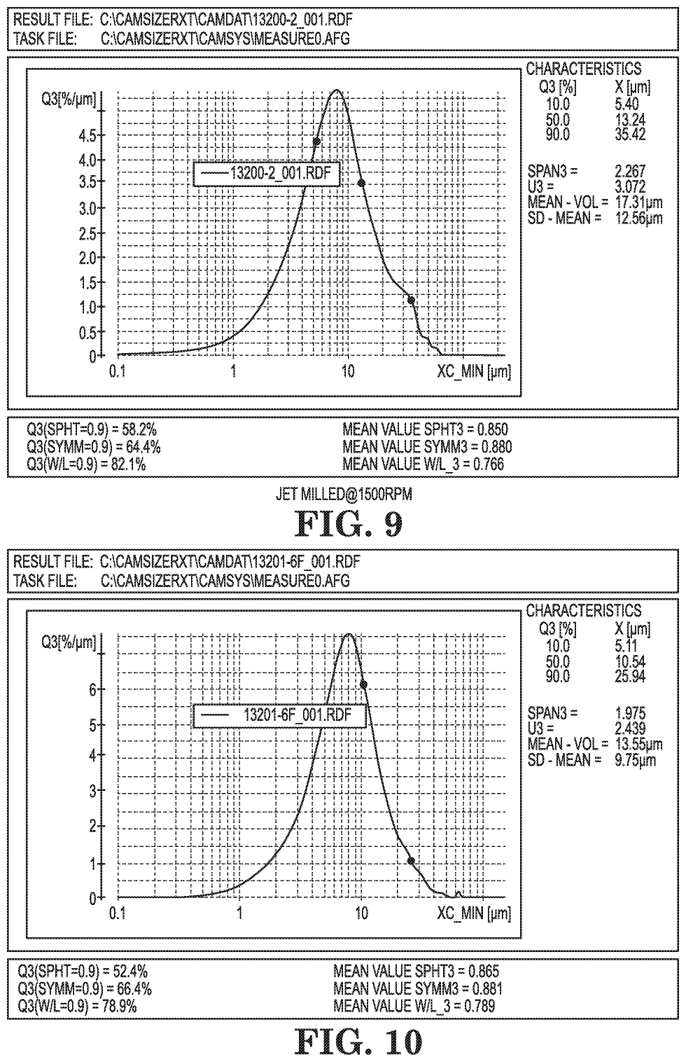

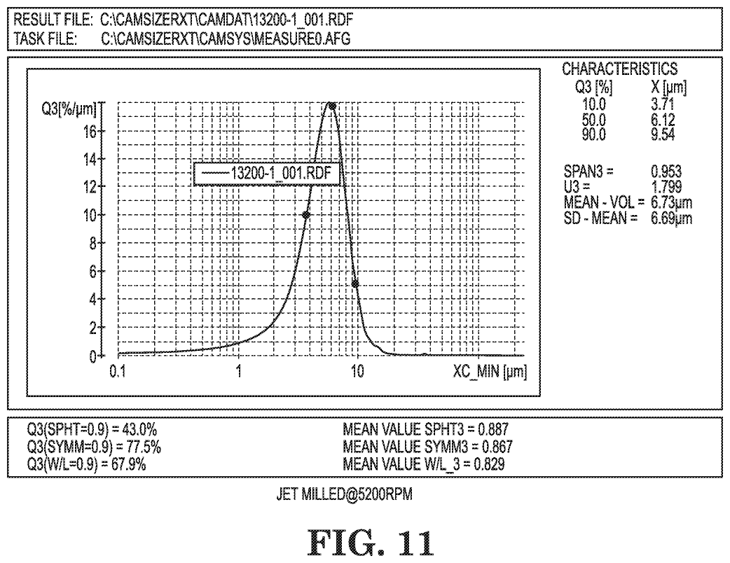

The particle size distributions are also shown in FIGS. 7-11, indicating standard deviation (SD), quarter 3 (Q3), span (SPAN3), volume (vol), and mean. FIG. 7 shows the size distribution for the unprocessed 90 PDI WF. FIG. 8 shows the size distribution for a sample from Example 1 produced in lab with a 90 PDI WF precursor material. FIG. 9 shows a sample jet milled at 1500 rpm, while FIG. 10 shows a sample that was jet milled at 500 rpm and air classified at 1500 rpm. FIG. 11 shows a sample that was jet milled at 5200 rpm. Overall, the jet milled 5200 rpm sample had much lower particle size and more discrete distribution compared to the other samples.

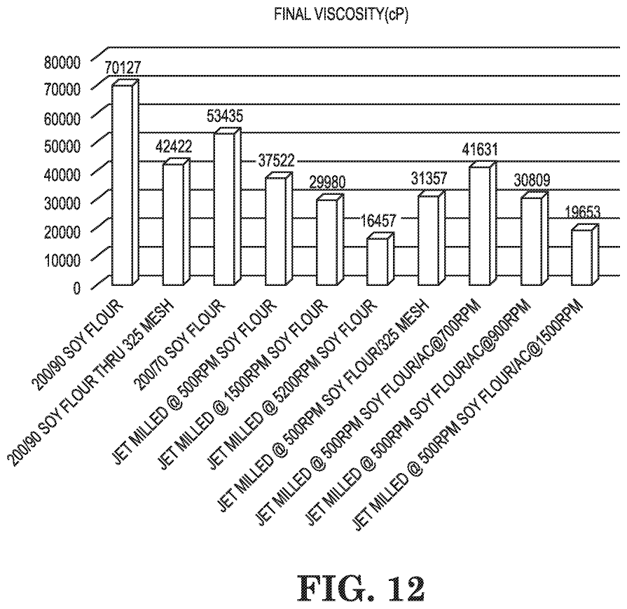

The viscosity of the samples in Example 3 was also studied by suspending the produced soy product in water. The change in particle size led to a dramatic change in viscosity similar to Example 1. There was a three to four fold reduction in viscosity compared to standard 200/70 and 200/90 soy flours. Viscosity is summarized in FIG. 12. In particular, sample 5 (90 PDI WF fines) performed the best, having a high protein percent and a low particle size.

Overall, Example 3 illustrates that a 90PDI WF could be milled and classified to produce a soy product for use in adhesive. Viscosities were generally lower with a similar solids content. Controllable differences in particle size distribution were observed with increased energy input at both the jet milling and air classification stages.

### Example 4—Jet Milling Trial

Example 4 included the use of varying speeds to grind the soy flake material, and analysis of protein content and viscosity. The results of Example 4 were compared to a standard 200/70 soy flour commonly used in a SoyAd® wood adhesive formula. Example 4 included testing of additional parameters in the jet milling (JM) and air classification (AC).

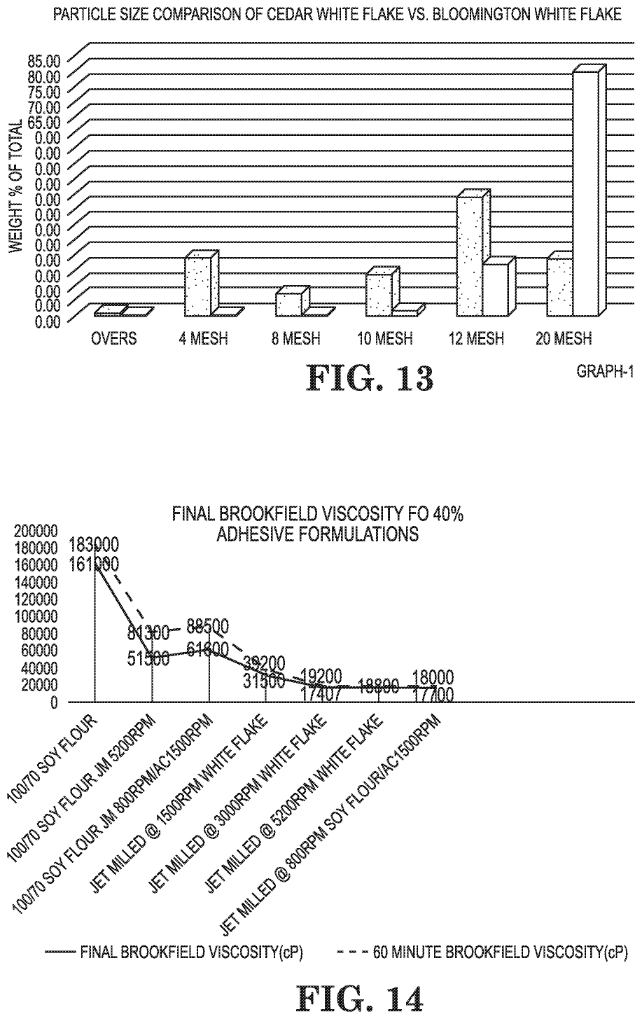

For Example 4, 90 PDI soy white flake (“90 soy flake”) (Cargill, Bloomington, Ill.) and 100 mesh, 70 PDI soy flour (“100/70 flour”) (Cargill, Cedar Rapids, Iowa) were processed. The samples were fractionated with mesh sizes of 8, 10, 12, and 20. A larger amount of hulls and non-white flake material was found in the soy white flake material. Weight fractions of both samples are shown in FIG. 13. Both samples were jet milled and analyzed.

Equipment used for Example 4 included a Model DPM-2 Fluidized Bed Jet Mill and Model 250 High Efficiency Centrifugal Air Classifier for processing of the samples. Analysis of the post-processed samples was done with Retsch Technology CamsizerXT instrument (Retsch Technology GmbH, Haan, Germany). The analysis included digital image analysis on shape and size information.

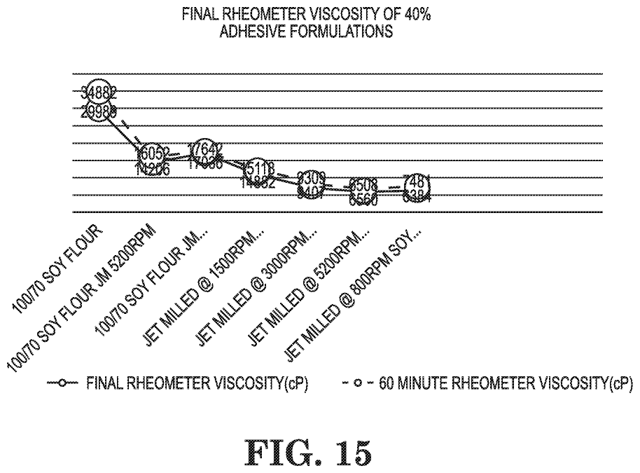

Rotational viscosity was measured with a Brookfield DV-1 viscometer with a #6 or #7 spindle at 10 rpm or 20 rpm. Viscosity measurements are shown in FIGS. 14-15.

Three jet milling and one air classification conditions were evaluated. Three jet milling parameters were controlled during the course of Example 3. First, the feed rate of the sample into the DPM-2 Jet Mill from a feed hopper was controlled. Second, fluid bed inlet pressure into the DPM-2 Jet Mill was controlled to keep the sample suspended within the unit. Third, the inlet air fan speed into the DPM-2 unit was controlled to monitor the amount of energy being injected into the system. These parameters were controlled in part due to higher rpms (i.e., more energy) resulting in smaller particle sizes.

Feed rate in the DPM-2 unit was monitored by internal static pressure to prevent too high of a product feed rate. The operator adjusted the feed rate, air pressure, and inlet fan speed to maintain internal static pressure in a steady state. Sampling occurred during these adjustments while particle size was monitored. Samples were jet milled at speeds of 800 rpm, 1500 rpm, and 5200 rpm, based on the results of Example 3.

After jet milling the sample material, the air classification process was initiated. Air classification was done with the sample material processed at 800 rpm. The milled sample materials were fed into the Model 250 Centrifugal Classifier controlled by a vibratory hopper. Inlet air pressure and rotor speed were maintained at a steady state. Samples were analyzed throughout the air classification to check progress and particle size. The results of Example 4 are summarized in Table 9 below.

TABLE 9
Summary of Example 4.

| | | | | | | | Mean |
|---|---|---|---|---|---|---|---|
| Sam- | | | | Air | | % | Particle |
| ple | Precursor | Jet | | Classi- | % | Protein | Size |
| ID | Material | Milling | | fied | MC | (Dry) | (Microns) |
| 1 | PDI 90 | N/A | | N/A | 6.79 | 49.70 | 1013.67 |
| | Soy Flake | | | | | | |
| 2 | PDI 90 | 5200 | rpm | N/A | 5.66 | 49.63 | 6.4 |
| | Soy Flake | | | | | | |
| 3 | PDI 90 | 4000 | rpm | N/A | 5.69 | 50.55 | 7.38 |
| | Soy Flake | | | | | | |
| 4 | PDI 90 | 3000 | rpm | N/A | 5.98 | 50.01 | 10.59 |
| | Soy Flake | | | | | | |
| 5 | PDI 90 | 2000 | rpm | N/A | 6.18 | 50.06 | 13.85 |
| | Soy Flake | | | | | | |
| 6 | PDI 90 | 1500 | rpm | N/A | 6.09 | 49.99 | 15.38 |
| | Soy Flake | | | | | | |
| 7 | PDI 90 | 800 | rpm | N/A | — | — | 28.56 |
| | Soy Flake | | | | | | |
| 8 | PDI 90 | 800 | rpm | 1500 rpm | 6.07 | 52.04 | 12.12 |
| | Soy Flake | | | | | | |
| 9 | PDI 90 | 800 | rpm | 1500 rpm | 6.24 | 46.66 | 46.59 |
| | Soy Flake | | | | | | |
| 10 | 100/70 | N/A | | N/A | 5.81 | 49.82 | 33.41 |
| | Soy Flour | | | | | | |
| 11 | 100/70 | 5200 | rpm | N/A | 4.60 | 49.18 | 6.54 |
| | Soy Flour | | | | | | |
| 12 | 100/70 | 800 | rpm | 1500 rpm | 6.14 | 50.21 | 11.53 |
| | Soy Flour | | | | | | |

Table 9 summarizes the results from Example 4. Table 9 shows the moisture content (% MC), dry protein (% Protein (Dry)), and mean particle size for each sample in Example 4. Table 9 shows particle sizes of the final soy product from the PDI 90 soy flake was from about 6 microns to about 12 microns. Overall, sample 8 (the PDI 90 soy flake jet milled at 800 rpm and air classified at 1500 rpm) performed the best, showing good protein levels and low particle size.

Samples from Example 4 were also suspended in water and analyzed for viscosity. Smaller particle size and higher protein levels led to lower viscosity at a given solids content. Viscosity of the suspensions increased over time within agitation. Trends in Viscosity are shown in FIGS. 14-15 and summarized in Table 10 below.

TABLE 10
Summary of Example 4 Viscosity Data at 40% solids.

| Sample | Final Brookfield Viscosity (cPs) |
|---|---|
| 10 | 161000 |
| 11 | 51500 |
| 12 | 61600 |
| 6 | 31500 |
| 4 | 19200 |
| 2 | 18300 |
| 8 | 17700 |

The viscosity measurements shown in Table 10 were taken with a Brookfield measurement instrument and method as described above. The viscosities shown in Table 10 represent the final viscosity of the samples after settling post testing (with an age time of about 20 to about 30 minutes). As discussed with reference to Table 10, the sample 10 was a conventional soy flour having a much higher viscosity at the same solids content relative samples 6, 4, 2, and 8, which were coarsely milled and fractionated soy flakes.

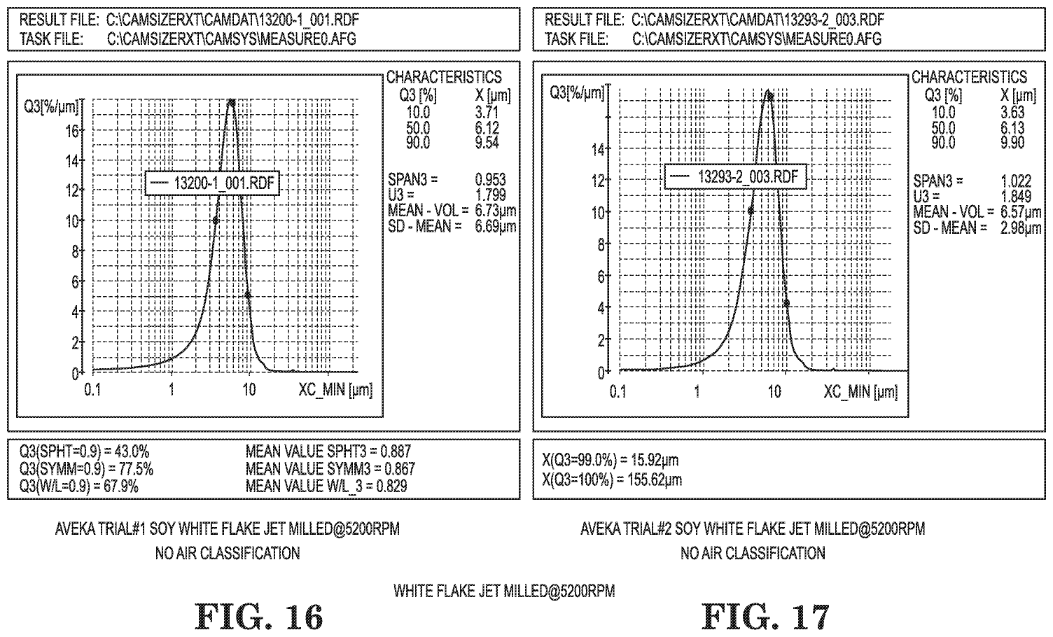

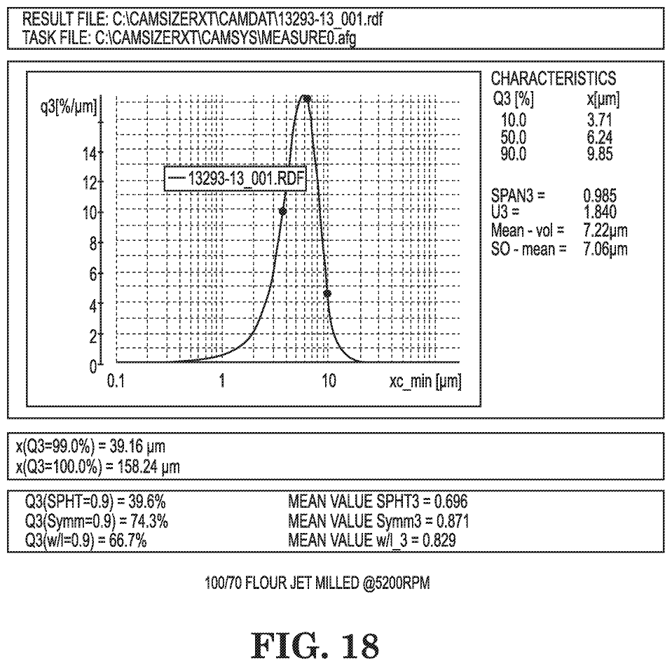

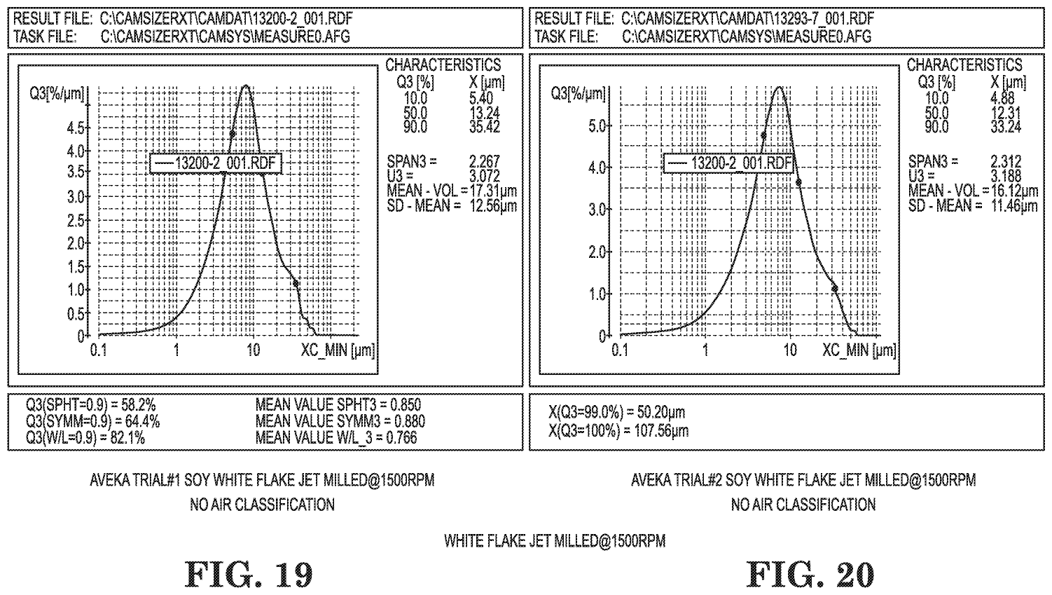

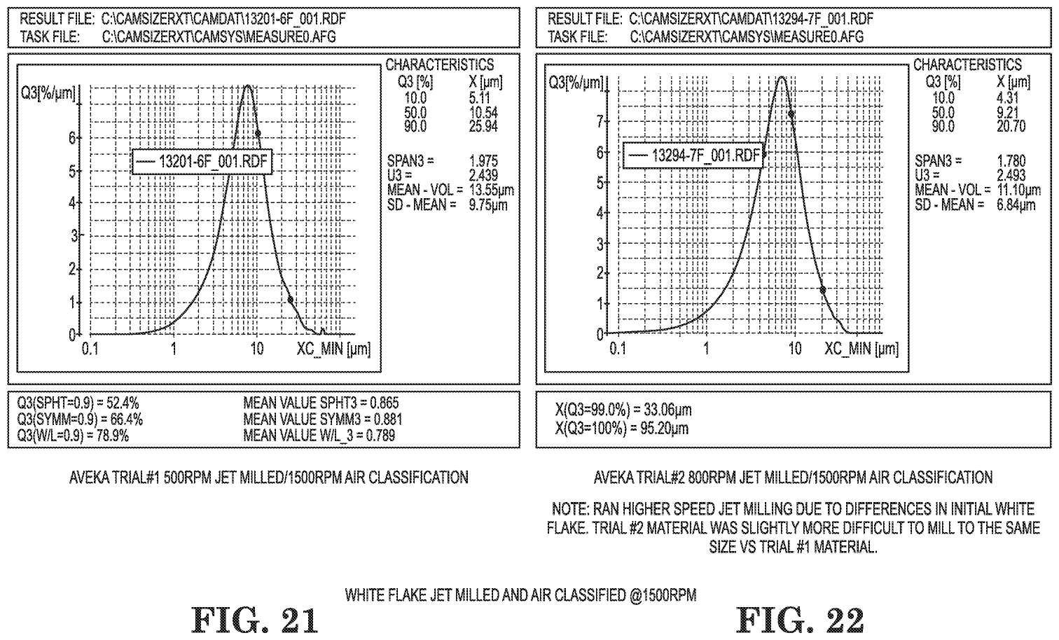

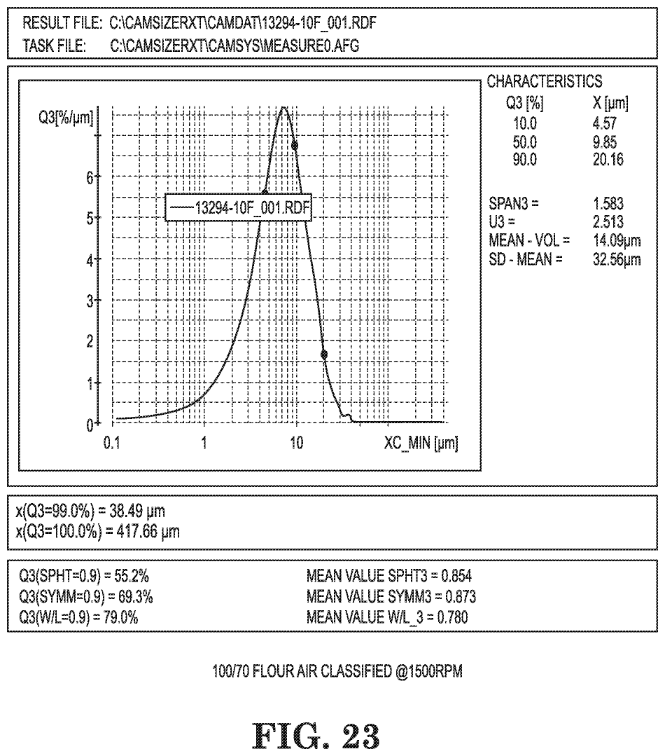

The samples from Example 4 were also analyzed for particle size data. The particle size distributions for Sample 2 (100/90 soy flake jet milled at 5200 rpm), Sample 12 (100/70 soy flour jet milled at 5200 rpm), Sample 6 (100/90 soy flake jet milled at 1500 rpm), Samples 8 and 9 (100/90 soy flake jet milled at 800 rpm and air classified at 1500 rpm), and Sample 12 (100/70 soy flour jet milled at 5200 rpm and air classified at 1500 rpm) are shown in FIGS. 16-23. The 100/90 soy flake Jet Milled 5200rm material (Sample 2) has a much lower particle size and more discrete distribution than other samples. This may show that jet milling alone boosted protein content similarly to 325-mesh sieving as in Example 1.

Overall, in Example 4, the jet milled 100/90 soy flake and 100/70 soy flour did not exhibit the same increase in protein content or lower viscosity. Increases in protein content of the jet milled PDI 90 soy flake sample were observed in multiple analyses. Difference in particle size distribution were observed with increased energy input at both the jet milling and air classification stages.

The results from Example 3 and Example 4 were similar. The materials produced were of comparable particle size and viscosity. In Example 4, the sample of PDI 90 soy flakes jet milled at 800 rpm and air classified at 1500 rpm had a 60.7% yield through the classifier, and a mean particle size of about 12.12 microns. By comparison, in Example 3, the soy flake sample jet milled at 500 rpm and air classified at 500 rpm had 61.84% yield through the classifier and a mean particle size of about 13.55 microns.

Examples 3 and 4 show a scaled-up production method of coarse ground and fractionated soy product produces a soy product with a high protein content that may be useful for adhesives.

### Example 5—Lab Screening of Plywood Adhesives

The milled and fractionated soy product was used to produce an adhesive that was, in turn, used to produce plywood panels in Examples 5 and 6. Several plywood adhesive evaluations were carried out in order to determine if a milled and fractionated soy product, as made in Examples 1-4, with higher protein levels, did improve the adhesive characteristics for plywood applications.

Plywood panels were made with ⅛″ (0.125 mm) Westcoast Fir core veneers (States Industries in Eugene, Oreg.). Seven ply panels were produced with these veneers, cut to slightly larger than 6″×6″ squares. Additionally, 1/64″ (0.0156 mm) Maple veneers were utilized for top and bottom faces on the 7-ply, ¾″ panel construction. The moisture level of the Fir cores was approximately 3%.

The prepared plywood panels were tested using a Carver laboratory 12 ton press with cold and hot press platens. During manufacture of the plywood panels, the hot platens were maintained at a constant temperature of about 113° C. (about 235° F.). Pressure across the platens was varied sample to sample. Specifically, samples were processed first with a 100 psi cold press for about 7 minutes, followed by a Hot Press at 130 psi press for about 7 minutes. Core temperatures for the 7-ply samples reached about 100° C. for two minutes under these conditions.

The adhesive formulation for the plywood panels used the produced milled and fractionated soy product (from Examples 1-4) in the SoyAd® formulation. The formulation included a polyamidoamine-polyepoxide polymer (PAE) labeled CA 1920A (20% solids) (Solenis, Wilminton, Del.). The ratio of soy flour to PAE resin was from about 6:1 to about 7:1 on a dry basis. The control for Example 5 was a 37% solids SoyAd formulation utilizing a standard commercial 200/70 soy flour. Additional additives to the formulation included 0.1% defoamer (MCA270, Hydrite, Inc.) and 0.25% sodium sulfite on a dry flour basis.

The adhesive was prepared by adding the defoamer and sodium sulfite to the commercial flour, followed by the water needed to hydrate the flour, e.g., between about 25 wt. % to about 50 wt. % or preferably about 30 wt. % to about 45 wt. % water. The mixture was mixed by hand until a thick paste was achieved with no visible dry soy flour particles visible. The PAE resin was then added to the paste and again mixed by hand until a smooth adhesive was achieved. Approximate total prep time for the adhesive was 20 minutes. Typically, the viscosity of SoyAd adhesives with this formulation and solids level are in the 100,000 cPs range.

For Example 5, the Jet Milled fraction labeled “JM5200” (i.e., jet milled at 2500 rpm) was used. This corresponds to the speed of the Jet Milling operation with no Air Classification. The average particle size of this experimental soy product was 12.5 microns. To compare the JM 5200 product with a standard 200/70 flour, several adhesives were formulated at 37%, 40%, 42%, and 45% solids, utilizing the same formulation components and levels described above. The water level of the adhesive was changed to produce the adhesives at different % solids. Due to the low viscosity of the JM 5200 product dispersions, it was possible to produce these experimental adhesives at substantially lower viscosities than the standard 200/70 flour. For example, even the 45% solids SoyAd formulation with the JM 5200 flour produced an adhesive that was far below 100,000 cPs.

After formulation of the adhesives, wood panels were prepared with the adhesives. The veneer panels were assembled targeting an as-is adhesive spread rate equating to 200-250 g/m² for each of the six veneer bond-lines. The panels were assembled in a conventional pattern with the new adhesive. With the prepared new adhesives, the adhesive spread rate when applied to the veneers was approximately 20-25% less, most likely due to the improved spread ability of the JM 5200 soy product formulations.

After the plywood panels were assembled, a 10 minute stand time with a 30 lb. weight was applied. After the stand time, a cold press was applied to the panels utilizing a Carver press targeting 7 minutes at an equivalent of 100 psi pressure. After the cold press, the panels were moved to the hot press platens, heated to a temperature of 235° F. (113° C.), for an additional 7 minutes at and equivalent of 135 psi pressure. After the hot press, the panels were allowed to cool and equilibrate in lab conditions overnight before testing.

Next, the plywood panels were prepared for testing. The ¾″ panels were cut into 3, 2″×5″ samples so there would be three testing results for each experimental panel.

To test adhesive strength, the panel samples were soaked in room temperature water for 4 hours, followed by 19 hours of drying at 50° C., following the wood industry standard test method for 3-cycle soak. After evaluating each bond line for delamination the sample panels were again soaked in water for 4 hours, followed by drying for 19 hours. The panels were then again soaked for 4 hours and dried for 19 hours, to complete the 3-cycle soak test.

After the third cycle was completed, each individual bond line was evaluated and rated on a 0-10 scale according to the wood industry guidelines. A “zero” bond line rating equates to absolutely no delamination in any of the bond lines. A “ten” bond line rating equates to a complete delamination of the bond line. A “six” rating is where there is greater than 2 inches (5 cm) of delamination in any bond line and is considered a bond failure. In our testing, each bond line is rated but then averaged to simplify the reporting of the 3-cycle soak rating for each test. For instance, a test condition would have six bond lines for each of the three sample panels for a total of eighteen bond lines to average. The lower the average rating, the better the delamination score.

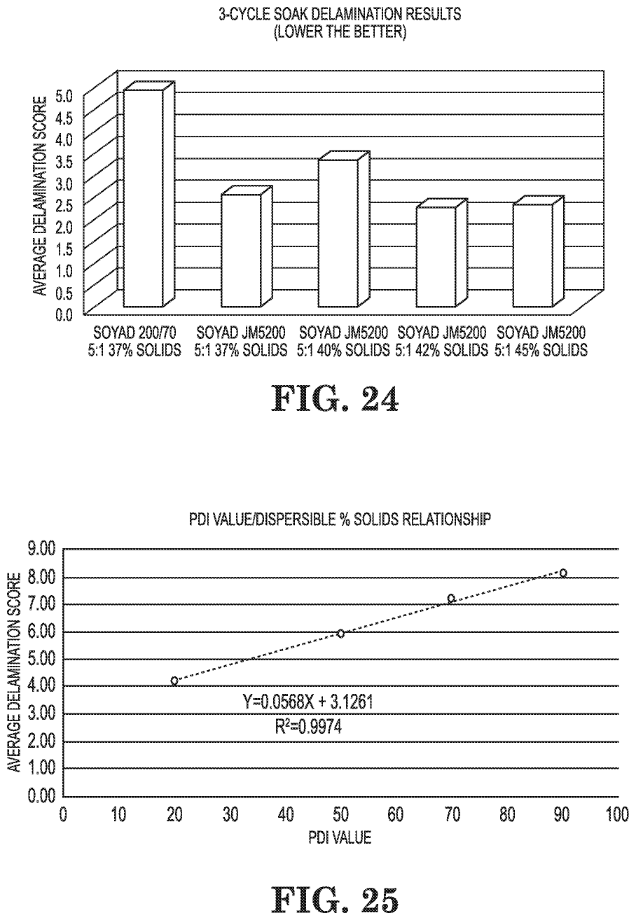

The resulting plywood product and corresponding adhesive strength was tested under standard 3-cycle soak tests, summarized in Table 11 below and shown in FIG. 24.

TABLE 11
Summary of Example 5 soak testing.

| | | | | | | | | Avg. |
|---|---|---|---|---|---|---|---|---|
| SAMPLE | BL1 | BL2 | BL3 | BL4 | BL5 | BL6 | SUM | Delam. |
| SoyAd 200/70 | 2.67 | 5.00 | 7.33 | 6.00 | 5.33 | 3.33 | 29.67 | 4.9 |
| 5:1 37% Solids | | | | | | | | |
| SoyAd JM5200 | 4.33 | 4.00 | 2.33 | 2.33 | 0.33 | 2.00 | 15.33 | 2.6 |
| 5:1 37% Solids | | | | | | | | |
| SoyAd JM5200 | 0.00 | 5.67 | 5.67 | 3.33 | 2.33 | 3.00 | 20.00 | 3.3 |
| 5:1 40% Solids | | | | | | | | |
| SoyAd JM5200 | 0.33 | 0.33 | 4.33 | 4.00 | 3.67 | 1.00 | 13.67 | 2.3 |
| 5:1 42% Solids | | | | | | | | |
| SoyAd JM5200 | 0.00 | 4.67 | 3.33 | 4.33 | 0.67 | 1.00 | 14.00 | 2.3 |
| 5:1 45% Solids | | | | | | | | |

In Table 11, samples using 200/70 flour (SoyAd 200/70), and jet milled samples from Examples 3 and 4 using the milled and fractionated soy product (SoyAd JM5200) with varying solids contents. Table 12 shows bond lines (BL) 1 through 6 scores and the total (SUM) of those scores, in addition to the average delamination of those samples (Avg. Delam.).

In general, there was a decline in delamination when comparing the new adhesive formulations with the commercial 200/70 soy flour based adhesive. Additionally, the formulations with a higher percent solids performed slightly better. The use of the JM 5200 soy product in the adhesive appeared to provide better soak resistance in the top and bottom maple face bonds, which are bond lines that tend to be difficult in conventional SoyAd formulations.

Overall, Example 5 shows adhesives can be made from the milled and fractionated soy product, such as the samples in Examples 1-4, to produce an adhesive having good tack (green adhesive strength), wet soak performance, and dry adhesive strength.

### Example 6—Delamination Testing with Automated Spreader

Additional testing of adhesives formulated with milled and fractionated soy product (“new adhesives,” as made in Examples 1-4) was done with an automated spreader in Example 6. Example 6 was a laboratory evaluation using an automated spreader, where several different flours from Example 3 were evaluated in the SoyAd formulation, in addition to several laboratory prepared experimental flours from Example 1.

In Example 6, the experimental flours evaluated were compared against a conventional SoyAd control with the standard 200/70 soy flour. The experimental flours from Example 3 were a combination of 90 PDI Jet Milled alone, and 90 PDI Jet Milled plus Air Classified samples.

The specific formulations of adhesives are shown in Tables 12-13 below:

TABLE 12
Adhesives Formulated in Example 6.

| | 1 | 2 | 3 | 4 | 5 | 6 | 7 | |
|---|---|---|---|---|---|---|---|---|
| Precursor | 70 PDI | 90 PDI | 70 PDI | 90 PDI | 90 PDI | 90 PDI | 90 PDI | |
| material | Soy | Soy | Soy | Soy | Soy | Soy | Soy | |
| | Flake | Flake | Flake | Flake | Flake | Flake | Flake | |
| Milled | CGC | JM | CGC | JM | JM | JM | JM | |
| | | 500 rpm | | 500 rpm | 5200 rpm | 5200 rpm | 500 rpm | |
| Fraction. | 325 M | AC | 325 M | AC | — | — | 325 | M |
| | | 700 rpm | | 700 rpm | | | | |
| Soy Amt. | 616.5 | 616.5 | 390.9 | 474.7 | 504.4 | 414.3 | 475.2 | |
| SMBS | 1.5 | 1.5 | 1.0 | 1.2 | 1.2 | 1.0 | 1.2 | |
| NaOH | 8.5 | 8.5 | 5.4 | 6.6 | 7.0 | 5.7 | 6.6 | |
| Defoam. | 1.0 | 1.0 | 0.8 | 0.9 | 1.0 | 0.9 | 0.9 | |
| PAE | 422.2 | 422.2 | 267.7 | 325.1 | 345.4 | 283.7 | 325.4 | |
| 1920A | | | | | | | | |
| Water | 538.8 | 538.8 | 459.8 | 559.6 | 521.0 | 519.9 | 490.8 | |
| Solids % | 42.5 | 42.5 | 38.0 | 38.0 | 40.0 | 37.0 | 40.0 | |
| Initial | 105,600 | 110,600 | 56,600 | 62,200 | 59,800 | 30,600 | 65,600 | |
| Viscosity | | | | | | | | |
| Density | 1.00 | 0.74 | 1.19 | 0.78 | 0.74 | 0.77 | 0.77 | |
| pH | 6.4 | 6.3 | 6.5 | NA | 6.6 | 6.7 | 6.5 | |
| Age | — | — | — | — | — | 22 min | 32 | min |
| Time | | | | | | | | |

| | | | | | | | |
|---|---|---|---|---|---|---|---|
| Final | 63,360 | 66,360 | 30,000 | 37,320 | 45,448 | 23,200 | 43,800 |
| Viscosity | | | | | | | |

TABLE 13
Adhesives Formulated in Example 6.

| | 8 | 9 | 10 | | 11 | 12 | 14 |
|---|---|---|---|---|---|---|---|
| Precursor | 90 PDI | 90 PDI | 50 PDI | | Soy | SoyAd | 90 PDI |
| material | Soy | Soy | Soy | | Flour | Control | Soy |
| | Flake | Flake | Flake | | 200/90 | | Flake |
| Milled | JM | JM | — | | | | JM |
| | 500 rpm | 1500 rpm | | | | | 500 rpm |
| Fraction. | AC | — | 325 | M | | | AC |
| | 1500 rpm | | | | | | 1500 rpm |
| Soy Amt. | 474.7 | 474.7 | 474.7 | | 474.7 | — | 518.6 |
| SMBS | 1.2 | 1.2 | 1.2 | | 1.2 | — | 1.3 |
| NaOH | 6.6 | 6.6 | 6.6 | | 6.6 | — | 6.6 |
| Defoam. | 0.9 | 0.9 | 0.9 | | 0.9 | — | 0.9 |
| PAE | 325.1 | 325.1 | 325.1 | | 325.1 | — | 267.7 |
| 1920A | | | | | | | |
| Water | 491.6 | 491.6 | 596.6 | | 491.6 | — | 526.5 |
| Solids % | 40.0 | 40.0 | 37.0 | | 40.0 | 39.3 | 42.0 |
| Initial | 36,600 | 52,400 | 70,400 | | 65,000 | 88,200 | 51,800 |
| Viscosity | | | | | | | |
| Density | 0.93 | 0.90 | 1.16 | | 0.89 | — | 0.90 |
| pH | 6.4 | 6.5 | 6.6 | | 6.4 | 6.8 | 6.7 |
| Age Time | 21 min | 23 min | 30 | min | 24 min | 69 min | 19 min |

| | | | | | | |
|---|---|---|---|---|---|---|
| Final | 24,200 | 32,200 | 45,200 | 40,200 | 79,400 | 33,600 |
| Viscosity | | | | | | |

Tables 12-13 shows samples 1-14. The amount of soy (Soy Amt. in grams) precursor material, sodium metabisulphite (SMBS), sodium hydroxide (NaOH, 25% solution), defoaming agent (Defoam.), the crosslinker (PAE 1920A), water, and other components used in the adhesive formulations. Samples 1-14 include soy sources such as jet milled (JM), air classified (AC), and 325-mesh sieved samples (325 M), and samples coarsely ground in the laboratory (CGC). The soy flour sample 11 was a conventional 200 mesh and 90 PDI soy flour, used as a control.

Tables 12-13 additionally show properties of these formulations, including initial viscosity (measured on a Brookfield instrument with spindle #7 at 20 rpm, listed in cPs), density (in g/mL), pH, viscosity age time (in minutes), and final viscosity after age time (in cPs).

The adhesive preparation process utilized a Kitchen Aid® mixer with the bread dough mixing attachment. The prescribed amount of water, anti-foam, and sodium bi-sulfite was added to the mixing bowl and the Kitchen Aid mixing head lowered into the mixture. The mixing speed is set to 3, and the soy product was added slowly until all of the soy product was wetted by the water. The mixing speed was then set to 10, and the components allowed to mix for 5 minutes. The CA 1920A crosslinker, available from Solenis, was then added and they were continually mixed for an additional 2 minutes. The pH was then adjusted to about 6.0 to 7.0 by the addition of the corresponding amount of 25% sodium hydroxide solution. Mixing was continued for an additional 3 minutes. At this point, the pH was measured with a calibrated pH meter to ensure the correct pH of the adhesive was reached.

In addition to the evaluation differences between the glue formulations, several different press conditions were tested in order to tease out properties of the experimental adhesives under mimicked production conditions.

For all adhesives, several 12″×12″×¾″ (30 cm×30 cm×2 cm) panels were produced and evaluated for dry bond and 3-cycle soak performance, as described above with reference to Example 5. The samples were additionally tested for viscosity, viscosity stability, spread ability, tack development, green strength, cure speed, press time, dry bond strength, and durability.

The panels, made of ⅛″ (0.3 cm) core fir veneers, face maple, and face oak, were freshly cut into 12″×12″ (30 cm×30 cm) squares from full panels taken from a commercial operation. Panels made in Example 6 used cold and hot press pressure based on typical commercial processes. The panels making process is summarized in Table 14 below.

TABLE 14
Panels in Example 6.

| | Spread | Stand | Cold | Hot |
|---|---|---|---|---|
| Panels | g/DGL | Minutes | Minutes | Minutes |
| 1 | 31-33 | 10 | 5 | 4 |
| 2 | 31-34 | 10 | 5 | 5.5 |
| 3 | 31-35 | 10 | 10 | 5.5 |
| 4 | 31-36 | 20 | 5 | 5.5 |
| 5 | 31-37 | 5 | NO | 5.5 |
| 6 | 31-38 | 10 | 5 | 4 |

The panels were made with a spread rate of about 32 to about 34 g/ft² and an assembly time of about 1 to 2 minutes of the lab spreader. The cold press applied about 93 psi during cold minutes. The hot press target temperature was 230° F. (110° C.). The equilibrium temperature was about 235° F. (113° C.).

An example plywood panel sample made and tested in Example 7, containing the new milled and fractionated soy product, are summarized in Table 15 below:

TABLE 15
Panel formation for Example 6.

| Grain | Sample | Wood | Lathe | Thickness |
|---|---|---|---|---|
| Direction | ID | Type | Checks | (in) |
| → | 1 | Maple | D | 0.02 |
| ↑ | 2 | FIR | D | 0.13 |
| → | 3 | FIR | D | 0.13 |
| ↑ | 4 | FIR | D | 0.13 |
| → | 5 | FIR | D | 0.13 |
| ↑ | 6 | FIR | D | 0.13 |
| → | 7 | Maple | U | 0.02 |
| | | | Total: | 0.67 |

The panels were made with a spread rate of from about 32 g/ft² to about 34 g/ft² and an assembly time of about 1 to 2 minutes with the laboratory spreader. The panels had a stand time of about 10 minutes with applied weight. Subsequently, the panel samples were cold pressed from about 5 to 10 minutes at about 93 psi. The panels samples were then hot pressed at about 110° C. with a press time of about 5.5 to about 7.0 seconds at 135 psi. The adhesives were tested with a standard 3-cycle soak tested and dry bond tested as described in reference to Example 5.

The produced adhesives and wood panels were tested for viscosity, spread ability, stand time tolerance, tack development (i.e., green strength), cure speed, press time, dry bond strength, and durability. 3-cycle soak testing and dry bond testing were assessed as discussed in Example 5.

Soak performance was comparable in all panels, including the conventional SoyAd control panels. Overall, the Jet Milled and Air Classified soy product samples from Example 3 performed significantly better in soak performance than did the Jet Milled samples from Example 3 alone. Out of the samples from Example 1, the 325 mesh fraction isolated after coarse milling of the PDI 90 white flake, performed very well in soak performance vs. the Example 3 samples that were Jet Milled alone, and comparable to the Example 3 Jet Milling/Air Classified samples.

Generally, the lower PDI value (70 and 50) samples generated in the laboratory screening process (Example 1) performed well in soak performance compared to the 90 PDI white flake Jet Milled samples alone (from Example 3) and were comparable to the 90 PDI Jet Milled/Air Classified products (from Example 3). Additionally, dry bond strength was good in all panels.

The new adhesive panels had lower viscosity, higher solids content, or both, particularly compared to the conventional SoyAd control panels. The solids content for the new adhesive panels increased by about 5 to 7% compared to the conventional SoyAd control. This can potentially reduce delamination.

The density of the new adhesives was about 0.75 to about 1.25 g/mL. This lower density may result from air entrainment (foaming) in the new adhesives that is not generally observed in conventional SoyAd controls with 200/70 PDI flour.

The new adhesive panels had longer stand time tolerance compared to the conventional SoyAd control panels. Additionally, the tack (i.e., green strength) for all panels was good, with little delamination observed from the cold press.

Overall, Example 6 shows adhesives can be made at a larger scale from the milled and fractionated soy product, such as the samples in Examples 1-4, to produce an adhesive having good tack (green adhesive strength), wet soak performance, and dry adhesive strength.

### Example 7—Analysis of Examples 1-6

The soy products and panels produced in Examples 1-7 were subject to further study regarding amount of protein, carbohydrates, and viscosity among other measurements, summarized in Table 16 below.

TABLE 16
Comparison of Produced Soy Product Samples.

| | | % | % Pr. | % | % | % Carbs |
|---|---|---|---|---|---|---|
| | Sample Description | Moisture | Dry | Lipid | Ash | Diff. |
| 1 | 90 PDI | 7.61 | 47.72 | 0.99 | 6.22 | 37.46 |
| | White Flake | | | | | |
| 2 | 100 mesh 90 PDI | 6.97 | 49.7 | 0.97 | 6.81 | 35.55 |
| | White Flake | | | | | |
| 3 | 90 PDI White Flake | 5.81 | 49.63 | 0.93 | 6.63 | 37.00 |
| | Jet Milled 5200 rpm | | | | | |
| 4 | 90 PDI White Flake | 6.92 | 52.04 | 1.03 | 7.15 | 32.86 |
| | Jet Milled 800 rpm | | | | | |
| | Air Classified | | | | | |
| | 1500 rpm | | | | | |
| 5 | 90 PDI White Flake | 6.30 | 46.66 | 1.46 | 6.43 | 39.15 |
| | Jet Milled 800 rpm | | | | | |
| | Air Classified | | | | | |
| | 1500 rpm | | | | | |
| | “Overs” | | | | | |
| 6 | 200 mesh 90 PDI | 5.89 | 49.71 | 1.00 | 6.50 | 36.90 |
| | Soy Flour | | | | | |
| 7 | 200 mesh 70 PDI | 5.85 | 49.83 | 1.00 | 6.74 | 36.58 |
| | Soy Flour | | | | | |
| 8 | 200 mesh 20 PDI | 6.70 | | 1.00 | 6.60 | |
| | Soy Flour | | | | | |
| 9 | PDI 70 | 7.24 | 53.68 | 1.00 | 6.70 | 31.38 |
| | White Flake Feed | | | | | |
| 10 | PDI 70 | 6.89 | 55.01 | 1.00 | 6.91 | 30.19 |
| | White Flake | | | | | |
| | 325 Mesh | | | | | |
| 11 | PDI 70 | 7.22 | 51.17 | 1.00 | 6.73 | 33.88 |
| | White Flake | | | | | |
| | 325 Mesh “Overs” | | | | | |
| 12 | PDI 90 | 6.57 | 50.85 | 1.00 | 6.73 | 34.85 |
| | White Flake | | | | | |
| | 325 Mesh | | | | | |
| 13 | 200 mesh 90 PDI | 6.47 | 49.67 | 1.00 | 6.91 | 35.95 |
| | Feed | | | | | |
| 14 | 200 mesh 90 PDI | 6.62 | 53.67 | 1.00 | 7.21 | 31.50 |
| | 625 Mesh | | | | | |

Table 16 shows the % moisture, % protein (Dry), % lipid, % ash, and % carbohydrates (calculated) for each of the samples. In general, the 90 PDI White Flake samples that was jet milled at 800 rpm and then air classified at 1500 rpm (Sample 5) performed well. Samples 1-5 were analyzed for % residual lipid utilizing a Soxlet extractor and methylene chloride as the extraction solvent. Because these analyses were all close to 1% residual lipid, the remaining samples were not analyzed but assumed to be 1% residual lipid.

Here, Samples 1 and 2 are defatted white soy flakes with a PDI of 90. Sample 2 has a 100 mesh rating.

Samples 3-5 have been processed as described in Examples 3 and 4 above. Sample 3 started with a 90 PDI white soy flake that was jet milled at 5200 rpm. Sample 4 started with a 90 PDI white soy flake that was jet milled at 800 rpm and air classified at 1500 rpm. Sample 5 contains the “overs” from Sample 4.

Samples 6-8 are commercially available soy flours at a 200 mesh rating, with varying PDI between 90, 70, and 20.

Sample 9 is an unprocessed soy white flake with PDI 70 that was used to create Samples 10, 11, and 12 with laboratory milling and fractionating as described in reference to Example 1. Sample 12 is the resulting soy product from processing of Sample 9 with a coffee grinder and 325 size mesh in a sieve stack. Sample 11 is the “overs” of Sample 12.

Samples 13 and 14 are based on commercially available soy flours with 200 mesh and 90 PDI. Sample 13 is the unprocessed flour, while Sample 14 is that flour run through a 325 size mesh as described in reference to Example 1.

Overall, Sample 4 (PDI 90 white flake jet milled at 800 rpm and air classified at 1500 rpm) and Sample 10 (PDI 70 white flake coarsely ground and sieved with 325 mesh) performed the best, having both high protein content and low particle size, particularly compared to traditional flours (i.e., Sample 14).

The percentages in Table 20 show percentages of the whole material through analysis methods described in detail with reference to Examples 1-7. In contrast, the dispersible percentages of these measurements can be seen in Table 17 below:

TABLE 17
Dispersible Percentages of Samples in Table 16.

| | | Dispersible | % | % | % Carbs |
|---|---|---|---|---|---|
| | Sample Description | % | Ash | Prot. | (Diff) |
| 1 | 90 PDI | 7.06 | 7.06 | 62.73 | 30.21 |
| | White Flake | | | | |
| 2 | 100 mesh 90 PDI | 7.49 | 7.49 | 62.2 | 30.31 |
| | White Flake | | | | |
| 3 | 90 PDI White Flake | 8.75 | 7.63 | 61.61 | 30.76 |
| | Jet Milled 5200 rpm | | | | |
| 4 | 90 PDI White Flake | 8.81 | 7.97 | 69.09 | 22.94 |
| | Jet Milled 800 rpm | | | | |
| | Air Classified 1500 rpm | | | | |
| 5 | 90 PDI White Flake | 7.64 | 6.91 | — | — |
| | Jet Milled 800 rpm | | | | |
| | Air Classified 1500 rpm | | | | |
| | “Overs” | | | | |
| 6 | 200 mesh 90 PDI | 8.15 | 7.71 | 63.1 | 29.19 |
| | Soy Flour | | | | |
| 7 | 200 mesh 70 PDI | 7.22 | 8.1 | 49.99 | 41.91 |
| | Soy Flour | | | | |
| 8 | 200 mesh 20 PDI | 4.23 | 18.64 | — | — |
| | Soy Flour | | | | |
| 9 | PDI 70 | — | — | — | — |
| | White Flake Feed | | | | |
| 10 | PDI 70 | 8.01 | 8.88 | 50.95 | 40.17 |
| | White Flake | | | | |
| | 325 Mesh | | | | |
| 11 | PDI 70 | — | — | — | — |
| | White Flake | | | | |
| | 325 Mesh “Overs” | | | | |
| 12 | PDI 90 | 8.64 | 8.86 | — | — |
| | White Flake | | | | |
| | 325 Mesh | | | | |
| 13 | 200 mesh 90 PDI | 8.35 | 8.08 | 60.63 | 31.29 |
| | Feed | | | | |
| 14 | 200 mesh 90 PDI | 8.86 | 7.89 | 60.13 | 31.98 |
| | 625 Mesh | | | | |

Here, the “feed” samples (i.e., samples 9, 13), are the measurements for the materials prior to processing.

Table 17 shows protein and carbohydrate amounts based on dispersible percent measurements. About 7-10% of the overall material for each sample was dispersible. That dispersible material was further analyzed for percent of proteins and ash in the dispersible material (i.e., dispersible % protein and dispersible % ash), with the dispersible % carbohydrates calculated from the difference. In general, the % protein in the dispersible material ranged from about 50 wt. % to about 70 wt. %.

PDI (protein dispersibility index) was calculated by a standard PDI procedure. A 400 mL Nalgene beaker fitted with a plastic stir rod was tared to 0.00 g. To this was added 10 g of the soy product sample and 20 g of DI water at room temperature. This was stirred by hand to a paste with no lumps. One drop of defoamer was added along with an additional 120 g of DI water. This was stirred by hand until the paste had completely dispersed in the water phase. The entire contents was transferred to a 150 ml stainless steel emulsifier cup with a rotor-type disperser, and capped. The motor was plugged into a rheostat set to 55 v and the mixture was allowed to stir for 10 minutes. After the mixing time was complete, the emulsion was transferred to a 400 mL tall form beaker and allowed to settle (approximately 15-20 minutes). The supernatant was siphoned off with a plastic dropper and 35 g was transferred to a 50 ml centrifuge tube. A second 50 mL centrifuge tube was charged with 35 g of DI water for balance. The sample was centrifuged at 2700 rpm for 10 minutes. The sample was removed and approximately 25 g of supernatant was added to a pre-weighed aluminum pan to four decimal points (0.0000 g). The sample and pan were weighed to four decimal points and recorded. The pan and sample were transferred to a forced air oven set to a temperature of 75° C., and this was allowed to dry for 24 hours. The pan and dry sample were removed from the oven and the weight recorded to four decimal points. The % dispersible were calculated and recorded. The dry sample was analyzed for nitrogen.

By comparison, dispersible calculations were generated on the new soy products. 10.00 g (dry basis) soy product was added to a 150 ml beaker fitted with a magnetic stir bar. To this was added 40° C. DI water to 100 g total weight, and this placed on a heating/stirring unit set to 40° C. The dispersion was stirred at 800 rpm for 45 minutes, ensuring that all of the soy product was hydrated. After heating and stirring, 45 ml of dispersion was transferred to a 50 ml capped centrifuge tube, with volume gradations, and this centrifuged at 500 rpm for 5 minutes. The tube was removed and approximately 20 g of supernatant was transferred to a previously dried and weighed (to 0.1 mg) ceramic ashing crucible plus ceramic top and the top+crucible+wet sample re-weighed to 0.1 mg. This was then placed in a forced air oven set to 110° C. for overnight drying. The amount of insoluble portion was also measured on the bottom of the centrifuge tube and recorded. After removal from the forced air oven, the top+crucible+dry sample was cooled and re-weighed to 0.1 mg to obtain a dispersible weight percent with a standard error between replicates of approximately +/−0.1%.

The sample was analyzed in duplicate for % Protein by the Elementary Nitrogen Analyzer. A factor of 5.71 was used to calculation the % Protein from the % Nitrogen. A second 45 mL sample from the heating and stirring reaction above was similarly dried in a forced air oven at 110° C. overnight, and the top, crucible, and dry sample was re-weighed to 0.1 mg. This sample was transferred to a cool ash oven and the heating ramped to 540° C. The samples were ashed for a minimum of 20 hours. After the ashing period, the samples were removed and cooled to room temperature. The top, crucible, and ash sample was re-weighed to 0.1 mg and expressed as a % of the dry sample weight. Subtracting the % Protein value and the % Ash value, the % Carbohydrate value was obtained by difference.

The dispersible calculations were fitted to a linear equation (where R²=0.9974) based on the test data and correlated to the reported PDI level. The percent of dispersible (by the method described above) material (Y) can be calculated with the equation (1) below based on the precursor material PDI (X):

Y=0.0568X+3.1261 (1)

The graph showing this relationship is shown as FIG. 25.

For bulk sample ashing, a ceramic ashing crucible and ceramic top were dried at 110° C. before use. The crucible and top were cooled to room temperature then weighed to 0.1 mg. Approximately 5 g of soy flour (as is basis) was added to the crucible and the top, crucible, and sample was re-weighed to 0.1 mg. The top, crucible, and sample were placed in a cool ashing oven, and the heat was ramped up to 540° C. The samples were ashed at 540° C. for a minimum of 20 hours. After the ashing period, the samples were removed from the ashing oven and cooled to room temperature. The top, crucible, and ash sample was re-weighed to 0.1 mg and expressed as a % of the dry sample weight obtained from the moisture analysis.

The % moisture in the bulk flour or white flake samples were determined on a HR73 Halogen Moisture Analyzer from Mettler-Toledo Corporation. A minimum of a 2 g sample was placed on the tared sample dish and ramped to a temperature of 130° C. After approximately 5 minutes the Analyzer automatically recorded the % moisture loss in the sample. Subtraction from 100 yields the dry % of the sample.

Overall, Sample 5 worked well in a wood adhesive application (see Examples 5 and 6), whereas Sample 3 (Jet Milled at 5200 rpm alone) did not show any improvement over the standard SoyAd formulation (Sample 7).

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present disclosure. Thus, it should be understood that although the present disclosure has been specifically disclosed by specific aspects and optional features, modification and variation of the concepts herein disclosed may be resorted to by those of ordinary skill in the art, and that such modifications and variations are considered to be within the scope of aspects of the present disclosure.

### Additional Embodiments

The following embodiments are provided, the numbering of which is not to be construed as designating levels of importance:

Embodiment 1 includes a soy product comprising: from about 50.0 wt. % to about 60.0 wt. % dry protein; less than 35.0 wt. % carbohydrates; and an increased amount of protein in a dispersible fraction of the soy product, compared to the amount of protein in a dispersible fraction of a defatted soy flake having the same starting protein dispersibility index.

Embodiment 2 includes Embodiment 1, wherein the soy product comprises greater than 51.0 wt. % dry protein.

Embodiment 3 includes any of Embodiments 1-2, wherein the soy product comprises greater than 52.0 wt. % dry protein.

Embodiment 4 includes any of Embodiments 1-3, wherein the soy product comprises less than 34.0 wt. % carbohydrates.

Embodiment 5 includes any of Embodiments 1-4, wherein the soy product comprises less than 33.0 wt. % carbohydrates.

Embodiment 6 includes any of Embodiments 1-5, wherein the increased amount of protein is at least 1.0%.

Embodiment 7 includes any of Embodiments 1-6, wherein the increased amount of protein is at least 2.0%

Embodiment 8 includes any of Embodiments 1-7, wherein the increased amount of protein is at least 3.0%

Embodiment 9 includes any of Embodiments 1-9, wherein the amount of protein increased is at least 1.0% relative to the amount of protein in the dispersible fraction of the defatted soy flake having the same starting protein dispersibility index.

Embodiment 10 includes any of Embodiments 1-9, wherein the amount of protein increased is at least 2.0% relative to the amount of protein in the dispersible fraction of the defatted soy flake having the same starting protein dispersibility index.

Embodiment 11 includes any of Embodiments 1-10, wherein the amount of protein increased is at least 3.0% relative to the amount of protein in the dispersible fraction of the defatted soy flake having the same starting protein dispersibility index.

Embodiment 12 includes any of Embodiments 1-11, wherein the defatted soy flake comprises lower than 1% measured oil.

Embodiment 13 includes and of Embodiments 1-12, wherein the soy product comprises lower than 1 wt. % measured oil.

Embodiment 14 includes a soy product comprising: from about 50.0 wt. % to about 60.0 wt. % dry protein; less than 35.0 wt. % carbohydrates; an increased amount of protein in a dispersible fraction of the soy product, compared to the amount of protein in a dispersible fraction of a defatted soy flake having the same starting protein dispersibility index; and wherein the soy product comprising one or more particles each having a particle size between about 20 microns and about 40 microns at the 90th percentile.

Embodiment 15 includes Embodiment 14, further comprising an increased amount of protein in a dispersible fraction of the soy product, compared to the amount of protein in a dispersible fraction of a defatted soy flake having the same starting protein dispersibility index

Embodiment 16 includes a soy product produced by a process, the process comprising: coarsely milling soy white flakes to provide a milled soy powder having a median 90th percentile particle size of about 50 microns to about 100 microns at the 90th percentile mean; and fractionating the milled soy powder to a soy product having greater than 50.0 wt. % dry protein, wherein the soy product have a median 90th percentile particle size particle size of about 20 microns to about 40 microns at the 90th percentile.

Embodiment 17 includes any of Embodiments 1-15, the soy powder having a median 90th percentile particle size particle size is about 60 microns to about 80 microns at the 90th percentile.

Embodiment 18 includes any of Embodiments 1-16, the soy product having a median 90th percentile particle size a particle size of about 25 microns to about 35 microns at the 90th percentile.

Embodiment 19 includes an aqueous adhesive composition comprising: the soy product of Embodiment 1; and a cross-linker crosslinking the soy product.

Embodiment 20 includes any of Embodiments 1-19, wherein when the adhesive composition is made to have about 30 wt. % to about 50 wt. % solids, the viscosity of that composition is less than about 100,000 cPs.

Embodiment 21 includes any of Embodiments 1-20, wherein the viscosity of that composition is less than about 50,000 cPs.

Embodiment 22 includes any of Embodiments 1-21, wherein the viscosity of that composition is less than about 20,000 cPs.

Embodiment 23 includes any of Embodiments 1-22, wherein the viscosity of that composition is less than about 10,000 cPs.

Embodiment 24 includes any of Embodiments 1-23, wherein the crosslinker is a reactive phosphorous oxide reagent a poly(glycidyl ether), a polyamidoamino epoxide (PAE) polymer, a multivalent metal oxide ion, a multivalent hydroxide, a multivalent organo alkoxide, or a multivalent halogen containing ion.

Embodiment 25 includes an article comprising: a wood product comprising plywood, hardwood plywood, external grade plywood flooring, engineered wood flooring, high density fiber board, medium density fiber board, or particle board, wherein the wood product is adhered with the adhesive of Embodiment 19.

Embodiment 26 includes a n article comprising: an engineered wood having at least one ply adhered together by the adhesive of Embodiment 19.

Embodiment 27 includes any of Embodiments 1-26, wherein the article passes a standard 3-cycle soak testing.

Embodiment 28 includes any of Embodiments 1-27, wherein the article passes a standard EN-314 test.

Embodiment 29 includes an article comprising: a particulate wood product comprising a plurality of wood particles, wherein the plurality of wood particles are adhered to each other by the adhesive of Embodiment 19.

Embodiment 30 includes a method of making a soy product, the method comprising: coarsely milling soy flakes to produce a milled soy powder; and fractionating the milled soy powder to produce the soy product of Embodiment 1.

Embodiment 31 includes any of Embodiments 1-30, wherein coarsely milling soy flakes comprises grinding with a double grinder.

Embodiment 32 includes an of the Embodiments 1-32, wherein coarsely milling soy flakes comprises milling with a hammermill, a Retsch mill, a pin mill, a jet milling machine, or other milling machines.

Embodiment 33 includes any of Embodiments 1-33, wherein fractionating comprises sieving the milled soy through a mesh with a rating of 325 or less.

Embodiment 34 includes any of Embodiments 1-34, wherein fractionating comprises air classification.

Embodiment 35 includes any of Embodiments 1-34, wherein the milled soy powder has a particle size between about 50 microns and about 100 microns at the 90th percentile.

1. (canceled)

2. The method of claim 30, wherein the soy product comprises greater than 51.0 wt. % dry protein.

3. The method of claim 30, wherein the soy product comprises greater than 52.0 wt. % dry protein.

4. The method of claim 30, wherein the soy product comprises less than 34.0 wt. % carbohydrates.

5. The method of claim 30, wherein the soy product comprises less than 33.0 wt. % carbohydrates.

6. The method of claim 30, wherein the increased amount of protein is at least 1.0%.

7. The method of claim 30, wherein the increased amount of protein is at least 2.0%

8. The method of claim 30, wherein the increased amount of protein is at least 3.0%

9. The method of claim 30, wherein the amount of protein is increased by least 1.0% relative to the amount of protein in the dispersible fraction of the defatted soy flake having the same starting protein dispersibility index.

10. The method of claim 30, wherein the amount of protein is increased by at least 2.0% relative to the amount of protein in the dispersible fraction of the defatted soy flake having the same starting protein dispersibility index.

11. The method of claim 30, wherein the amount of protein is increased by at least 3.0% relative to the amount of protein in the dispersible fraction of the defatted soy flake having the same starting protein dispersibility index.

12.-18. (canceled)

19. An aqueous adhesive composition comprising:
the soy product of produced from claim 30; and
a cross-linker crosslinking the soy product.

20. The aqueous adhesive composition of claim 19, wherein when the adhesive composition is made to have about 30 wt. % to about 50 wt. % dry solids, the viscosity of that composition is less than about 100,000 cPs.

21. The aqueous adhesive composition of claim 20, wherein the viscosity of that composition is less than about 50,000 cPs.

22. The aqueous adhesive composition of claim 20, wherein the viscosity of that composition is less than about 20,000 cPs.

23. The aqueous adhesive composition of claim 20, wherein the viscosity of that composition is less than about 10,000 cPs.

24-29. (canceled)

30. A method of making a soy product, the method comprising:
coarsely milling soy flakes to produce a milled soy powder; and
fractionating the milled soy powder to produce the soy product comprising from about 50.0 wt. % to about 60.0 wt. % dry protein;
less than 35.0 wt. % carbohydrates; and
an increased amount of protein in a dispersible fraction of the soy product, compared to the amount of protein in a dispersible fraction of a defatted soy flake having the same starting protein dispersibility index.

31. The method of claim 30, wherein coarsely milling soy flakes comprises grinding with a double grinder.

32. The method of claim 30, wherein coarsely milling soy flakes comprises milling with a hammermill, a Retsch mill, a pin mill, a jet milling machine, or other milling machines.

33. The method of claim 30, wherein fractionating comprises sieving the milled soy through a mesh with a rating of 325 or less.

34. The method of claim 30, wherein fractionating comprises air classification.

35. The method of claim 30, wherein the milled soy powder has a mean 90th percentile particle size between about 50 microns and about 100 microns.
